(19) United States
(12) Patent Application Publication
(10) Pub. No.: US 2024/0228626 A1
(43) Pub. Date: Jul. 11, 2024

(54) ANTI-NKG2D ANTIBODIES AND USES THEREOF

(71) Applicant: IMMUNORIZON LTD., Rehovot (IL)

(72) Inventors: Oren BOGIN, Tel-Aviv (IL); Liat DASSA, Tel Aviv (IL)

(73) Assignee: IMMUNORIZON LTD., Rehovot (IL)

(21) Appl. No.: 18/558,679

(22) PCT Filed: May 2, 2022
(86) PCT No.: PCT/IL22/50453

§ 371 Date: Nov. 2, 2023

Related U.S. Application Data
(60) Provisional application No. 63/183,635, filed on May 4, 2021.

(51) Int. Cl. C07K 16/28; A61K 39/00; A61P 37/04

(52) U.S. Cl. CPC C07K 16/2851; A61P 37/04; A61K 2039/505; C07K 2317/31; C07K 2317/565

(57) ABSTRACT

The present disclosure describes a number of anti-NKG2D antibodies. In some embodiments, anti-NKG2D antibodies comprising certain complementarity determining regions (CDRs) are disclosed. The anti-NKG2D antibodies disclosed herein can be used to treat various diseases such as cancer.

### CROSS REFERENCE TO RELATED APPLICATION(S)

This application is a 35 U.S.C. 371 National Phase Entry Application from PCT/IL2022/050453, filed on May 2, 2022, which claims the benefit of U.S. Provisional Application No. 63/183,635, filed on May 4, 2021, the disclosures of which are incorporated therein in their entirety by reference.

### SEQUENCE LISTING STATEMENT

The instant application contains a Sequence Listing, which has been submitted electronically in ASCII format and is hereby incorporated by reference in its entirety. Said ASCII copy, created on Oct. 31, 2023 is named P-604135-PC_SL.txt and is 21,204 bytes in size.

### FIELD OF DISCLOSURE

The present disclosure relates in general to antibodies. In one embodiment, the present disclosure describes the making and uses of antibodies against NKG2D.

### BACKGROUND

NKG2D (Nature Killer Group 2, member D) is an activating cell surface protein that is predominantly expressed on cytotoxic immune cells. NKG2D is abundantly present on all NK cells, NKT cells, and subsets of γδ T cells. While naïve human CD8⁺ T cells express NKG2D, in mice they upregulate its expression only after activation. CD4⁺ T cells generally do not express NKG2D even after activation, but in humans its expression can be induced under certain pathological conditions, such as Crohn's disease juvenile-onset lupus and cytomegalovirus infection. The molecular structure of NKG2D allows it to bind a number of structurally different MHC-I-like ligands. NKG2D ligands have in common that under homeostatic conditions their expression is generally low. In contrast, upon cellular stress, such as infection or oncogenic transformation, their expression can be highly induced. In humans, the NKG2D ligands are MICA, MICB, and six members of the ULBP family.

NKG2D is a homodimer of two disulfide-linked transmembrane proteins, with very short intracellular domains that do not have signaling properties. In mice, NKG2D uses the adaptor molecules DAP10 and DAP12 to relay its signaling, whereas in humans NKG2D associates exclusively with DAP10. DAP10 and DAP12 initiate different signaling cascades. DAP10 possesses a YINM motif which allows binding p85 of phosphatidylinositol-3 kinase (PI3K). In addition, DAP10 binds Grb2, which associates with Vav1. DAP12 contains an immune receptor tyrosine-based activation motif, which is phosphorylated by Src-kinases upon NKG2D triggering. This event allows binding and activation of the tyrosine kinases, Syk and Zap70.

NKG2D plays an important role in the recognition and elimination of potentially dangerous cells. It has been shown to mediate immune responses against tumors, virally infected cells, and organ transplants. For this reason, NKG2D was originally thought to predominantly mediate direct cytotoxicity in response to the encounter of ligand on stressed target cells. However, in most cases, NKG2D is only able to mediate immune cell activation if it occurs within an inflammatory context. Both NK and T cells generally require a secondary signal before NKG2D is able to mediate a measurable effect. The primary function of NKG2D therefore appears to be regulation of signaling through other receptors. Its unique feature is that it is able to both inhibit and potentiate signaling of a large number of receptors in multiple ontologically distinct immune cell subsets and during different stages of the life cycle of immune cells, such as hematopoietic development, priming, and effector responses.

NKG2D has great potential as a therapeutic target, since it has the potency to enhance cytolytic immune responses against important diseases, such as cancer. In view of the important roles of NKG2D, there is a need to develop anti-NKG2D antibodies to study the therapeutic uses of NKG2D.

### SUMMARY

In one embodiment, the present disclosure provides a number of anti-NKG2D antibodies. In one embodiment, each of the anti-NKG2D antibodies comprises three complementarity determining regions (CDRs) on a heavy chain (HCDR1, HCDR2, and HCDR3) and three CDRs on a light chain (LCDR1, LCDR2, and LCDR3), wherein
the HCDR1, HCDR2, and HCDR3 comprises the amino acid sequences of SEQ ID NOs: 12-14, and the LCDR1, LCDR2, and LCDR3 comprises the amino acid sequences of SEQ ID NOs: 16-18; or
the HCDR1, HCDR2, and HCDR3 comprises the amino acid sequences of SEQ ID NOs:20-22, and the LCDR1, LCDR2, and LCDR3 comprises the amino acid sequences of SEQ ID NOs:24-26; or
the HCDR1, HCDR2, and HCDR3 comprises the amino acid sequences of SEQ ID NOs:28-30, and the LCDR1, LCDR2, and LCDR3 comprises the amino acid sequences of SEQ ID NOs:32-34; or
the HCDR1, HCDR2, and HCDR3 comprises the amino acid sequences of SEQ ID NOs:36-38, and the LCDR1, LCDR2, and LCDR3 comprises the amino acid sequences of SEQ ID NOs:40-42.

In one embodiment, each of the anti-NKG2D antibodies comprises a heavy chain variable region and a light chain variable region, wherein the amino acid sequences for the heavy chain variable region and the light chain variable region can be one of the following pairs: SEQ ID NOs: 11 and 15; SEQ ID NOs: 19 and 23; SEQ ID NOs:27 and 31; or SEQ ID NOs:35 and 39.

In one embodiment, the present disclosure provides a composition comprising a pharmaceutically acceptable carrier and an anti-NKG2D antibody disclosed herein.

The present disclosure also provides polynucleotide sequences encoding the anti-NKG2D antibodies disclosed herein, as well as vectors and host cells comprising such polynucleotide sequences.

In one embodiment, the anti-NKG2D antibodies disclosed herein can be used to treat diseases such as cancer, autoimmune diseases, GvHD, viral infection or bacterial infection. In another embodiment, the anti-NKG2D antibodies disclosed herein can be used to treat diseases associated with NKG2D. In another embodiment, the anti-NKG2D antibodies disclosed herein can be used to treat diseases associated with over-expression of NKG2D.

These and other aspects of the anti-NKG2D antibodies will be appreciated from the ensuing descriptions of the figures and detailed description of the anti-NKG2D antibodies.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the anti-NKG2D antibodies and uses thereof are herein described, by way of example only, with reference to the accompanying drawings. With specific reference now to the drawings in detail, it is stressed that the particulars shown are by way of example and for purposes of illustrative discussion of embodiments of the anti-NKG2D antibodies and uses thereof. In this regard, the description taken with the drawings makes apparent to those skilled in the art how embodiments of the anti-NKG2D antibodies and uses thereof may be practiced.

FIGS. 1A-1B show binding of mice serum test bleed following immunization (TB) or pre-bleed before immunization (PB) to recombinant human NKG2D-ECD-Fc (extracellular domain of NKG2D fused to human Fc). FIG. 1A shows results of ELISA binding of serum from Balb/c mice (mice #8741, 8741, 8743, 8744, 8745) or SJL mice (mice #8746, 8747, 8748, 8749, 8750). FIG. 1B shows FACS binding of serum from Balb/c mice (mice #8741, 8741, 8743, 8744, 8745) and SJL mice (mice #8746, 8747, 8748, 8749, 8750) against human-NKG2D/Dap10 over-expressed in CHO cells, or against cyno-NKG2D/Dap10 over-expressed in CHO cells, while no reactivity was observed on CHO parental cells. hIgG1: negative human IgG control. mIgG1: negative mouse IgG control. NKG2D Ab: positive control hIgG mAb.

FIG. 2 lists the identities of selected mouse hybridoma clones.

FIG. 3 shows the binding of selected purified mAbs (mAb001 to mAb020) to the NKG2D-ECD-Fc antigen by ELISA.

FIGS. 4A-4C show FACS binding of selected purified mAbs (mAb001 to mAb020) to CHO cells over-expressing human or cyno NKG2D/Dap10. FIG. 4A shows binding to CHO cells over-expressing human NKG2D/Dap10. FIG. 4B shows binding to CHO cells over-expressing cyno NKG2D/Dap10. FIG. 4C presents binding to primary NK cells, showing dose dependent binding in all mAbs (100 nM, left bar for each mAb; or 10 nM, right bar for each mAb) with various mean fluorescence intensity (MFI). Tab KYK2.0 is a positive anti NKG2D-human IgG1 antibody with LALA P329G mutation in the Fc. Human IgG1LALA P328G is a human IgG negative control Ab with LALA P329G mutations in the Fc.

FIG. 5 summarizes the epitope binning analysis performed by ELISA, showing 4 different groups as follows: Group1 (rectangle continuous line) gather mAb001, 002, 003, 004, 005, 006, 008, 011, 013, 015, 017, 018, 020. Group 2 (circle continuous line) gather mAb007, 010, 014. Group 3 (rectangle dashed line) gather mAb012, 016, 019. Group4 (circle dashed line) gather mAb009

FIGS. 6A-6B present the activation of NK cells upon binding of the purified anti-NKG2D mAbs (mAb001 to mAb020) at 100 nM (left bar for each mAb) or 10 nM (right bar for each mAb)). FIG. 6A shows accumulation of CD107a activation marker. APC CD107A is anti CD107A antibody staining without anti NKG2D mAb clones binding, APC mIgG is a negative control mouse IgG antibody. IM-1060 is a positive control Tribody, IM-1091 is a negative control Tribody. FIG. 6B shows IFN-gamma secretion. IM-1091 is a negative control Tribody.

FIG. 7 shows the inhibition of NKG2D-MICA (MICA—MHC Class I Polypeptide-Related Sequence A) interaction by selected purified mAbs (mAb001 to mAb020).

FIG. 8A shows one embodiment of a Tribody structure. FIG. 8B shows one embodiment of a ProTribody structure. FIG. 8C lists the identities of various Tribody molecules.

FIG. 9 shows Tribody proteins (IM1244 to IM1251) characterization by SDS-PAGE.

| Ab ID | Ab Name | Heavy Chain | Light Chain |
|---|---|---|---|
| IM1244 | CD3x5T4x NKG2D-cl-1 (L-H) | h1F3.5Fd- | 1F3.1λ-5T4IM53_HuCL16-13 |
| | | NKG2D_cl1_ScFv(L-H) | (BM7) |
| IM1245 | CD3x5T4x NKG2D-cl-1 (H-L) | h1F3.5Fd- | 1F3.1λ-5T4IM53_HuCL16-13 |
| | | NKG2D_cl1_ScFv(H-L) | (BM7) |
| IM1246 | CD3x5T4x NKG2D-cl-9 (L-H) | h1F3.5Fd- | 1F3.1λ-5T4IM53_HuCL16-13 |
| | | NKG2D_cl9_ScFv(L-H) | (BM7) |
| IM1247 | CD3x5T4x NKG2D-cl-9 (H-L) | h1F3.5Fd- | 1F3.1λ-5T4IM53_HuCL16-13 |
| | | NKG2D_cl9_ScFv(H-L) | (BM7) |
| IM1248 | CD3x5T4x NKG2D-cl-10 (L-H) | h1F3.5Fd- | 1F3.1λ-5T4IM53_HuCL16-13 |
| | | NKG2D_cl10_ScFv(L-H) | (BM7) |
| IM1249 | CD3x5T4x NKG2D-cl-10 (H-L) | h1F3.5Fd- | 1F3.1λ-5T4IM53_HuCL16-13 |
| | | NKG2D_cl10_ScFv(H-L) | (BM7) |
| IM1250 | CD3x5T4x NKG2D-cl-19 (L-H) | h1F3.5Fd- | 1F3.1λ-5T4IM53_HuCL16-13 |
| | | NKG2D_cl19_ScFv(L-H) | (BM7) |
| IM1251 | CD3x5T4x NKG2D-cl-19 (H-L) | h1F3.5Fd- | 1F3.1λ-5T4IM53_HuCL16-13 |
| | | NKG2D_cl19_ScFv(H-L) | (BM7) |
| IM1060 | CD3/5T4/NKG2D(VL-VH) | h1F3.5Fd- KYK-2.0_ScFv(L-H) | h1F3.1λ-ScFv(L-H) |
| IM1091 | CD3/5T4/NKG2D mut(VL-VH) | h1F3.5Fd- KYK-2.0 | h1F3.1λ-ScFv(L-H) |
| | | mut_ScFv(L-H) | |

FIGS. 10A-10H show Tribody proteins (IM1244 to IM1251) characterization by analytical SEC.

FIG. 11 shows binding of Tribody proteins (IM1244 to IM1251, IM1060 and IM1091) to NK2G protein by ELISA. IM-1060 is a positive control Tribody, IM-1091 is a negative control Tribody.

FIGS. 12A-12B show binding of Tribody proteins (IM1244 to IM1251, IM1060 and IM1091) to CHO cells over-expressing human NK2G protein (FIG. 12A) or CHO cells over-expressing cyno NKG2D (FIG. 12B). IM-1060 is a positive control Tribody, IM-1091 is a negative control Tribody

FIG. 13 shows NK cell activation by Tribody proteins (IM1245, IM1246, IM1249, IM1250, IM1060 and IM1091) at 300 nM, 60 nM, 12 nM and 2.4 nM (bars, left to right). E+T APC-CD107 is CD107a staining in the absence of the NKG2D mAbs. E APC-CD107 is CD107 staining in the absence of the NKG2D mAbs and target cells.

### DETAILED DESCRIPTION

The present disclosure presents isolated anti-NKG2D antibodies, wherein unique CDR sequences of anti-NKG2D mAb are provided within a humanized framework (chimeric antibody; humanized antibody). In addition, incorporation of the NKG2D antigen binding regions of these anti-NKG2D antibodies into multi-valent antibody construct is demonstrated. The anti-NKG2D antibodies disclosed herein could potentially be used as an immunotherapeutic treatment for a medical condition, for example cancer.

As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with a target antigen. By “specifically binding” is meant that the binding is selective for the antigen of interest and can be discriminated from unwanted or nonspecific interactions. For example, an antibody is said to specifically bind a NKG2D epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹²M. In some embodiments, the equilibrium dissociation constant may be in the range of ≤10⁻⁵ M to 10⁻¹²M.

Half maximal effective concentration (EC₅₀) refers to the concentration of a drug, antibody or toxicant which induces a response halfway between the baseline and maximum responses after a specified exposure time. In some embodiments, the response comprises a binding affinity. In some embodiments, the response comprises a functional response for example an agonistic response. A skilled artisan would appreciate that as used herein in certain embodiments, the EC₅₀ measurement of an anti-NKG2D antibody disclosed herein provides a measure of a half-maximal binding of the anti-NKG2D antibody to the NKG2D antigen (EC₅₀ binding). The skilled artisan would appreciate that as used herein in certain embodiments, the EC₅₀ measurement of an anti-NKG2D antibody disclosed herein provides a measure of a half-maximal effective concentration of the anti-NKG2D antibody to induce an agonist response (EC₅₀ functional agonism).

In some embodiments, EC₅₀ comprises the concentration of antibody required to obtain a 50% agonist response that would be observed upon antibody binding. In certain embodiments, a measure of EC₅₀ is commonly used as a measure of a drug's potency and may in some embodiments, reflect the binding of the antibody to the receptor. In some embodiments, anti-NKG2D antibodies having nanomolar EC₅₀ binding concentration measurements comprise tight binding anti-NKG2D antibodies. In some embodiments, anti-NKG2D antibodies having nanomolar EC₅₀ functional agonism concentration measurements comprise functionally effective agonistic antibodies. In certain embodiments, an anti-NKG2D antibody disclosed herein comprises a tight binder to the NKG2D molecule. In certain embodiments, an anti-NKG2D antibody disclosed herein comprises an agonist for the NKG2D molecule. In certain embodiments, an anti-NKG2D antibody disclosed herein comprises a tight binding agonist for the NKG2D molecule.

In some embodiments, the binding EC₅₀ of an anti-NKG2D antibody is in the nanomolar range. In some embodiments, the binding EC₅₀ of an anti-NKG2D antibody comprises a range of about 0.05-100 nM. In some embodiments, the binding EC₅₀ of an anti-NKG2D antibody comprises a range of about 0.05-50 nM. In some embodiments, the binding binding EC₅₀ of an anti-NKG2D antibody comprises a range of about 0.05-20 nM. In some embodiments, the binding EC₅₀ of an anti-NKG2D antibody comprises a range of about 0.05-10 nM. In some embodiments, the binding EC₅₀ of an anti-NKG2D antibody comprises a range of about 0.1-100 nM. In some embodiments, the binding EC₅₀ of an anti-NKG2D antibody comprises a range of about 0.1-50 nM. In some embodiments, the binding EC₅₀ of an anti-NKG2D antibody comprises a range of about 0.1-20 nM. In some embodiments, the binding EC₅₀ of an anti-NKG2D antibody comprises a range of about 0.1-10 nM. In some embodiments, the binding EC₅₀ of an anti-NKG2D antibody comprises a range of about 1-100 nM. In some embodiments, the binding EC₅₀ of an anti-NKG2D antibody comprises a range of about 1-20 nM. In some embodiments, the binding EC₅₀ of an anti-NKG2D antibody comprises a range of about 20-40 nM. In some embodiments, the binding EC₅₀ of an anti-NKG2D antibody comprises a range of about 40-60 nM. In some embodiments, the binding EC₅₀ of an anti-NKG2D antibody comprises a range of about 60-80 nM. In some embodiments, the binding EC₅₀ of an anti-NKG2D antibody comprises a range of about 80-100 nM. In some embodiments, the binding EC₅₀ of an anti-NKG2D antibody comprises a range of about 1-40 nM. In some embodiments, the binding EC₅₀ of an anti-NKG2D antibody comprises a range of about 1-60 nM. In some embodiments, the binding EC₅₀ of an anti-NKG2D antibody comprises a range of about 1-80 nM. In some embodiments, the binding EC₅₀ of an anti-NKG2D antibody comprises a range of about 1-50 nM. In some embodiments, the binding EC₅₀ of an anti-NKG2D antibody comprises a range of about 0.05-5 nM. In some embodiments, the binding EC₅₀ of an anti-NKG2D antibody comprises a range of about 0.1-5 nM. In some embodiments, the binding EC₅₀ of an anti-NKG2D antibody comprises a range of about 0.05-20 nM.

In some embodiments, the binding EC₅₀ of an anti-NKG2D antibody comprises a range of about 0.05-5 nM. In some embodiments, the binding EC₅₀ of an anti-NKG2D antibody comprises a range of about 0.1-5 nM. In some embodiments, the binding EC₅₀ of an anti-NKG2D antibody comprises a range of about 1-5 nM. In some embodiments, the binding EC₅₀ of an anti-NKG2D antibody comprises a range of about 0.05-10 nM. In some embodiments, the binding EC₅₀ of an anti-NKG2D antibody comprises a range of about 0.1-10 nM. In some embodiments, the binding EC₅₀ of an anti-NKG2D antibody comprises a range of about 1-10 nM. In some embodiments, the binding EC₅₀ of an anti-NKG2D antibody comprises a range of about 5-10 nM. In some embodiments, the binding EC₅₀ of an anti-NKG2D antibody comprises a range of about 0.05-15 nM. In some embodiments, the binding EC₅₀ of an anti-NKG2D antibody comprises a range of about 0.01-15 nM. In some embodiments, the binding EC₅₀ of an anti-NKG2D antibody comprises a range of about 1-15 nM.

In some embodiments, the EC₅₀ measuring functional agonism is referred herein as the function EC₅₀, having all the same qualities. In some embodiments, the functional EC₅₀ of an anti-NKG2D antibody is in the nanomolar range. In some embodiments, the functional EC₅₀ of an anti-NKG2D antibody comprises a range of about 0.05-100 nM. In some embodiments, the functional EC₅₀ of an anti-NKG2D antibody comprises a range of about 0.05-50 nM. In some embodiments, the functional EC₅₀ of an anti-NKG2D antibody comprises a range of about 0.05-20 nM. In some embodiments, the functional EC₅₀ of an anti-NKG2D antibody comprises a range of about 0.05-10 nM. In some embodiments, the functional EC₅₀ of an anti-NKG2D antibody comprises a range of about 0.1-100 nM. In some embodiments, the functional EC₅₀ of an anti-NKG2D antibody comprises a range of about 0.1-50 nM. In some embodiments, the functional EC₅₀ of an anti-NKG2D antibody comprises a range of about 0.1-20 nM. In some embodiments, the functional EC₅₀ of an anti-NKG2D antibody comprises a range of about 0.1-10 nM. In some embodiments, the functional EC₅₀ of an anti-NKG2D antibody comprises a range of about 1-100 nM. In some embodiments, the functional EC₅₀ of an anti-NKG2D antibody comprises a range of about 1-20 nM. In some embodiments, the functional EC₅₀ of an anti-NKG2D antibody comprises a range of about 20-40 nM. In some embodiments, the functional EC₅₀ of an anti-NKG2D antibody comprises a range of about 40-60 nM. In some embodiments, the functional EC₅₀ of an anti-NKG2D antibody comprises a range of about 60-80 nM. In some embodiments, the functional EC₅₀ of an anti-NKG2D antibody comprises a range of about 80-100 nM. In some embodiments, the functional EC₅₀ of an anti-NKG2D antibody comprises a range of about 1-40 nM. In some embodiments, the functional EC₅₀ of an anti-NKG2D antibody comprises a range of about 1-60 nM. In some embodiments, the functional EC₅₀ of an anti-NKG2D antibody comprises a range of about 1-80 nM. In some embodiments, the functional EC₅₀ of an anti-NKG2D antibody comprises a range of about 1-50 nM. In some embodiments, the functional EC₅₀ of an anti-NKG2D antibody comprises a range of about 0.05-5 nM. In some embodiments, the functional EC₅₀ of an anti-NKG2D antibody comprises a range of about 0.1-5 nM. In some embodiments, the functional EC₅₀ of an anti-NKG2D antibody comprises a range of about 0.05-20 nM.

In some embodiments, the functional EC₅₀ of an anti-NKG2D antibody comprises a range of about 0.05-5 nM. In some embodiments, the functional EC₅₀ of an anti-NKG2D antibody comprises a range of about 0.1-5 nM. In some embodiments, the functional EC₅₀ of an anti-NKG2D antibody comprises a range of about 1-5 nM. In some embodiments, the functional EC₅₀ of an anti-NKG2D antibody comprises a range of about 0.05-10 nM. In some embodiments, the functional EC₅₀ of an anti-NKG2D antibody comprises a range of about 0.1-10 nM. In some embodiments, the functional EC₅₀ of an anti-NKG2D antibody comprises a range of about 1-10 nM. In some embodiments, the functional EC₅₀ of an anti-NKG2D antibody comprises a range of about 5-10 nM. In some embodiments, the functional EC₅₀ of an anti-NKG2D antibody comprises a range of about 0.05-15 nM. In some embodiments, the functional EC₅₀ of an anti-NKG2D antibody comprises a range of about 0.01-15 nM. In some embodiments, the functional EC₅₀ of an anti-NKG2D antibody comprises a range of about 1-15 nM.

As used herein, the term “antibody” encompasses an antibody fragment or fragments that retain binding specificity including, but not limited to, IgG, heavy chain variable region (VH), light chain variable region (VL), Fab fragments, F(ab′)2 fragments, scFv fragments, Fv fragments, a nanobody, minibodies, diabodies, triabodies, tetrabodies, and single domain antibodies (see, e.g., Hudson and Souriau, Nature Med. 9: 129-134 (2003)). Also encompassed are humanized, primatized, and chimeric antibodies as these terms are generally understood in the art. In some embodiments, an antibody disclosed herein comprises a precursor construct wherein the antigen binding site may be blocked by a regulatory domain, wherein exposure of the binding site comprise a regulated exposure based on environmental conditions, for example but not limited to, exposure to a tumor micro-environment.

As used herein, the term “heavy chain variable region” may be used interchangeably with the term “VH domain” or the term “VH”, having all the same meanings and qualities. As used herein, the term “light chain variable region” may be used interchangeably with the term “VL domain” or the term “VL”, having all the same meanings and qualities. A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions are more highly conserved than the CDRs, and form a scaffold to support the CDRs. Similarly, a skilled artisan would also recognize that a “light chain variable region” or “VL” with regard to an antibody encompasses the fragment of the light chain that contains three CDRs interposed between framework regions.

As used herein, the term “complementarity determining region” or “CDR” refers to the hypervariable region(s) of a heavy or light chain variable region. Proceeding from the N-terminus, each of a heavy or light chain polypeptide has three CDRs denoted as “CDR1,” “CDR2,” and “CDR3”. Crystallographic analysis of a number of antigen-antibody complexes has demonstrated that the amino acid residues of CDRs form extensive contact with a bound antigen. Thus, the CDR regions are primarily responsible for the specificity of an antigen-binding site. In one embodiment, an antigen-binding site includes six CDRs, comprising the CDRs from each of a heavy and a light chain variable region.

As used herein, the term “framework region” or “FR” refers to the four flanking amino acid sequences which frame the CDRs of a heavy or light chain variable region. Some FR residues may contact bound antigen; however, FR residues are primarily responsible for folding the variable region into the antigen-binding site. In some embodiments, the FR residues responsible for folding the variable regions comprise residues directly adjacent to the CDRs. Within FRs, certain amino residues and certain structural features are very highly conserved. In this regard, all variable region sequences contain an internal disulfide loop of around 90 amino acid residues. When a variable region folds into an antigen binding site, the CDRs are displayed as projecting loop motifs that form an antigen-binding surface. It is generally recognized that there are conserved structural regions of FR that influence the folded shape of the CDR loops into certain “canonical” structures regardless of the precise CDR amino acid sequence. Furthermore, certain FR residues are known to participate in non-covalent interdomain contacts which stabilize the interaction of the antibody heavy and light chains.

An antibody may exist in various forms or having various domains including, without limitation, a complementarity determining region (CDR), a variable region (Fv), a VH domain, a VL domain, a single chain variable region (scFv), and a Fab fragment.

A person of ordinary skill in the art would appreciate that a scFv is a fusion polypeptide comprising the variable heavy chain (VH) and variable light chain (VL) regions of an immunoglobulin, connected by a short linker peptide. The linker may have, for example, 10 to about 25 amino acids.

A skilled artisan would also appreciate that the term “Fab” with regard to an antibody generally encompasses that portion of the antibody consisting of a single light chain (both variable and constant regions) bound to the variable region and first constant region of a single heavy chain by a disulfide bond, whereas F(ab′)₂ comprises a fragment of a heavy chain comprising a VH domain and a light chain comprising a VL domain.

In some embodiments, an antibody encompasses whole antibody molecules, including monoclonal and polyclonal antibodies. In some embodiments, an antibody encompasses an antibody fragment or fragments that retain binding specificity including, but not limited to, variable heavy chain (VH) fragments, variable light chain (VL) fragments, Fab fragments, F(ab′)₂ fragments, scFv fragments, Fv fragments, minibodies, diabodies, triabodies, and tetrabodies.

In one embodiment, the anti-NKG2D antibodies disclosed herein can be incorporated as part of a bispecific antibody. As it is generally known in the art, bispecific antibody is a recombinant protein that includes antigen-binding fragments of two different monoclonal antibodies, and is thereby capable of binding two different antigens. In some embodiments, the anti-NKG2D antibodies disclosed herein are bi-valent for NKG2D. In some embodiments, the anti-NKG2D antibodies disclosed herein are monovalent for binding NKG2D.

In one embodiment, the anti-NKG2D antibodies disclosed herein can be incorporated as part of a multi-specific antibody. In one embodiment, a multi-specific antibody is a recombinant protein that includes antigen-binding fragments of at least two different monoclonal antibodies, such as two, three or four different monoclonal antibodies. In one embodiment, the anti-NKG2D antibodies disclosed herein can be incorporated as part of a tri-specific antibody.

In some embodiments, bispecific, tri-specific, or multi-specific antibodies are used for cancer immunotherapy by simultaneously targeting more than one antigen target, for example, a cytotoxic T cell (CTL) as well as a tumor associated antigen (TAA), or simultaneously targeting a CTL receptor component such as CD3, an effector natural killer (NK) cells, and a tumor associated antigen (TAA).

### Anti-NKG2D Antibodies

The present disclosure provides a number of anti-NKG2D antibodies. In one embodiment, each of the anti-NKG2D antibodies comprises a set of three complementarity determining regions (CDRs) on a heavy chain (HCDR1, HCDR2, and HCDR3) and a set of three CDRs on a light chain (LCDR1, LCDR2, and LCDR3).

In one embodiment, the HCDR1, HCDR2, and HCDR3 comprises the amino acid sequences of SEQ ID NOs: 12-14, and the LCDR1, LCDR2, and LCDR3 comprises the amino acid sequences of SEQ ID NOs: 16-18.

In one embodiment, the HCDR1, HCDR2, and HCDR3 comprises the amino acid sequences of SEQ ID NOs:20-22, and the LCDR1, LCDR2, and LCDR3 comprises the amino acid sequences of SEQ ID NOs:24-26.

In one embodiment, the HCDR1, HCDR2, and HCDR3 comprises the amino acid sequences of SEQ ID NOs:28-30, and the LCDR1, LCDR2, and LCDR3 comprises the amino acid sequences of SEQ ID NOs:32-34.

In one embodiment, the HCDR1, HCDR2, and HCDR3 comprises the amino acid sequences of SEQ ID NOs:36-38, and the LCDR1, LCDR2, and LCDR3 comprises the amino acid sequences of SEQ ID NOs:40-42.

In another embodiment, the anti-NKG2D antibodies comprises heavy chain and light chain CDR sequences that are at least 80% (e.g., at least 85%, 90%, 95%, 96%, 97%, 98%, or 99%) identical to the amino acid sequences set forth above.

In one embodiment, each of the anti-NKG2D antibodies presented herein comprises a heavy chain variable region (VH) and a light chain variable region (VL), wherein the amino acid sequences for the heavy chain variable region and the light chain variable region can be one of the following pairs: SEQ ID NOs: 11 and 15; SEQ ID NOs: 19 and 23; SEQ ID NOs:27 and 31; or SEQ ID NOs:35 and 39. In another embodiment, the anti-NKG2D antibodies comprise VH and VL sequences that are at least 80% (e.g., at least 85%, 90%, 95%, 96%, 97%, 98%, or 99%) identical to the amino acid sequences set forth above.

In one embodiment, in view of the sequences for the heavy chain variable regions and light chain variable regions disclosed herein, one of ordinary skill in the art would readily employ standard techniques known in the art to construct an anti-NKG2D scFv.

In certain embodiments, the present disclosure provides polypeptides comprising the VH and VL domains which could be dimerized under suitable conditions. For example, the VH and VL domains may be combined in a suitable buffer and dimerized through appropriate interactions such as hydrophobic interactions. In another embodiment, the VH and VL domains may be combined in a suitable buffer containing an enzyme and/or a cofactor which can promote dimerization of the VH and VL domains. In another embodiment, the VH and VL domains may be combined in a suitable vehicle that allows them to react with each other in the presence of a suitable reagent and/or catalyst.

In certain embodiments, the VH and VL domains may be contained within longer polypeptide sequences that may include for example, but not limited to, constant regions, hinge regions, linker regions, Fc regions, or disulfide binding regions, or any combination thereof. A constant domain is an immunoglobulin fold unit of the constant part of an immunoglobulin molecule, also referred to as a domain of the constant region (e.g. CH1, CH2, CH3, CH4, Ck, Cl). In some embodiments, the longer polypeptides may comprise multiple copies of one or both of the VH and VL domains generated according to the method disclosed herein; for example, when the polypeptides generated herein are used to forms a diabody or a triabody.

In one embodiment, the anti-NKG2D antibody presented herein can be an IgG, a Fv, a scFv, a Fab, a F(ab′)2, a minibody, a diabody, a triabody, a nanobody, a bispecific antibody, a tri-specific antibody, a multi-specific antibody, or a single domain antibody. For example, the anti-NKG2D antibody can be IgG such as IgG1, IgG2, IgG3, or IgG4. In some embodiments, the anti-NKG2D antibody comprises an IgG1. In some embodiments, the anti-NKG2D antibody comprises an IgG2. In some embodiments, the anti-NKG2D antibody comprises an IgG3. In some embodiments, the anti-NKG2D antibody comprises an IgG4.

In one embodiment, the present disclosure provides antibodies that bind with high affinity to NKG2D. In one embodiment, binding affinity is calculated by a modification of the Scatchard method as described by Frankel et al. (Mol. Immunol., 16:101-106, 1979). In another embodiment, binding affinity is measured by an antigen/antibody dissociation rate. In another embodiment, binding affinity is measured by a competition radioimmunoassay. In another embodiment, binding affinity is measured by ELISA. In another embodiment, antibody affinity is measured by flow cytometry.

In one embodiment, the present disclosure also provides isolated polynucleotide sequence encoding the heavy chain and light chain CDRs as described herein. In another embodiment, the present disclosure also provides a vector comprising such polynucleotide sequences. In view of the amino acid sequences disclosed herein, one of ordinary skill in the art would readily construct a vector or plasmid to encode for the amino acid sequences. In another embodiment, the present disclosure also provides a host cell comprising the vector provided herein. Depending on the uses and experimental conditions, one of skill in the art would readily employ a suitable host cell to carry and/or express the above-mentioned polynucleotide sequences.

In one embodiment, the present disclosure also provides isolated polynucleotide sequence encoding the heavy chain and light chain variable regions as described herein. In another embodiment, the present disclosure also provides a vector comprising such polynucleotide sequences. In view of the amino acid sequences disclosed herein, one of ordinary skill in the art would readily construct a vector or plasmid to encode for the amino acid sequences. In another embodiment, the present disclosure also provides a host cell comprising the vector provided herein. Depending on the uses and experimental conditions, one of skill in the art would readily employ a suitable host cell to carry and/or express the above-mentioned polynucleotide sequences.

The table below (Table 1) lists the names of the anti-NKG2D antibody clones, and the SEQ ID NOs for the corresponding VH, VL and CDRs.

TABLE 1
SEQ ID NOs, Clone Names, and Ab Component thereof.

| SEQ ID NO: | Name | Ab Component |
|---|---|---|
| | NKG2D | |
| 1 | NKG2D (KYK2.0) | VH |
| 2 | | CDR1 |
| 3 | | CDR2 |
| 4 | | CDR3 |
| 5 | | VL |
| 6 | | CDR1 |
| 7 | | CDR2 |
| 8 | | CDR3 |
| 9 | NKG2D(KYK2.0)mut | VH |
| 10 | | VL |
| 11 | IM, NKG2D mouse hybridoma cl- 1 | VH |
| 12 | | CDR1 |
| 13 | | CDR2 |
| 14 | | CDR3 |
| 15 | | VL |
| 16 | | CDR1 |
| 17 | | CDR2 |
| 18 | | CDR3 |
| 19 | IM, NKG2D mouse hybridoma cl- 9 | VH |
| 20 | | CDR1 |
| 21 | | CDR2 |
| 22 | | CDR3 |
| 23 | | VL |
| 24 | | CDR1 |
| 25 | | CDR2 |
| 26 | | CDR3 |
| 27 | IM, NKG2D mouse hybridoma cl- 10 | VH |
| 28 | | CDR1 |
| 29 | | CDR2 |
| 30 | | CDR3 |
| 31 | | VL |
| 32 | | CDR1 |
| 33 | | CDR2 |
| 34 | | CDR3 |
| 35 | IM, NKG2D mouse hybridoma cl- 19 | VH |
| 36 | | CDR1 |
| 37 | | CDR2 |
| 38 | | CDR3 |
| 39 | | VL |
| 40 | | CDR1 |
| 41 | | CDR2 |
| 42 | | CDR3 |

### Compositions for Use

In one embodiment, the present disclosure also provides a composition comprising the anti-NKG2D antibody disclosed herein and a pharmaceutically acceptable carrier. Pharmaceutically acceptable carriers of use are well-known in the art. For example, Remington's Pharmaceutical Sciences, by E. W. Martin, Mack Publishing Co., Easton, PA, 15th Edition, 1975, describes compositions and formulations suitable for pharmaceutical delivery of the antibodies disclosed herein. In one embodiment, the composition comprises anti-NKG2D antibodies that comprise a set of three complementarity determining regions (CDRs) on a heavy chain (HCDR1, HCDR2, and HCDR3) and a set of three CDRs on a light chain (LCDR1, LCDR2, and LCDR3).

In some embodiments of compositions, the HCDR1, HCDR2, and HCDR3 comprises the amino acid sequences of SEQ ID NOs: 12-14, and the LCDR1, LCDR2, and LCDR3 comprises the amino acid sequences of SEQ ID NOs: 16-18.

In some embodiments of compositions, the HCDR1, HCDR2, and HCDR3 comprises the amino acid sequences of SEQ ID NOs:20-22, and the LCDR1, LCDR2, and LCDR3 comprises the amino acid sequences of SEQ ID NOs:24-26.

In some embodiments of compositions, the HCDR1, HCDR2, and HCDR3 comprises the amino acid sequences of SEQ ID NOs:28-30, and the LCDR1, LCDR2, and LCDR3 comprises the amino acid sequences of SEQ ID NOs:32-34.

In some embodiments of compositions, the HCDR1, HCDR2, and HCDR3 comprises the amino acid sequences of SEQ ID NOs:36-38, and the LCDR1, LCDR2, and LCDR3 comprises the amino acid sequences of SEQ ID NOs:40-42.

In another embodiment, the composition comprises anti-NKG2D antibodies having heavy chain and light chain CDR sequences that are at least 80% (e.g., at least 85%, 90%, 95%, 96%, 97%, 98%, or 99%) identical to the amino acid sequences set forth above.

In another embodiment, the composition comprises anti-NKG2D antibodies having one of the following pairs of heavy chain variable region and light chain variable region: SEQ ID NOs:11 and 15; SEQ ID NOs: 19 and 23; SEQ ID NOs:27 and 31; or SEQ ID NOs:35 and 39.

In another embodiment, the composition comprises anti-NKG2D antibodies having VH and VL sequences that are at least 80% (e.g., at least 85%, 90%, 95%, 96%, 97%, 98%, or 99%) identical to the amino acid sequences set forth above.

In some embodiments of compositions, the antibodies disclosed herein can be in the form of a conjugate. As used herein, a “conjugate” is an antibody or antibody fragment (such as an antigen-binding fragment) covalently linked to an effector molecule or a second protein (such as a second antibody). The effector molecule can be, for example, a drug, toxin, therapeutic agent, detectable label, protein, nucleic acid, lipid, nanoparticle, carbohydrate or recombinant virus. An antibody conjugate can also be referred to as an “immunoconjugate.” When the conjugate comprises an antibody linked to a drug (e.g., a cytotoxic agent), the conjugate can be referred to as an “antibody-drug conjugate”. Other antibody conjugates include, for example, multi-specific (such as bispecific or trispecific) antibodies and chimeric antigen receptors (CARs).

A composition comprising the anti-NKG2D antibody or an antigen-binding fragment thereof can be administered to a subject (e.g. a human or an animal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. The pharmaceutical compositions may be prepared by methodology well known in the pharmaceutical art.

The pharmaceutical compositions comprising the antibodies or antigen-binding fragments thereof disclosed herein can be administered (e.g., to a mammal, a cell, or a tissue) in any suitable manner depending on whether local or systemic treatment is desired. For example, the composition can be administered topically (e.g. ophthalmically, vaginally, rectally, intranasally, transdermally, and the like), orally, by inhalation, or parenterally (including by intravenous drip or subcutaneous, intracavity, intraperitoneal, intradermal, or intramuscular injection). Topical intranasal administration refers to delivery of the compositions into the nose and nasal passages through one or both of the nares. The composition can be delivered by a spraying mechanism or droplet mechanism, or through aerosolization. Alternatively, administration can be intratumoral, e.g. local or intravenous injection.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve preparation of a slow-release or sustained-release system so as to maintain a constant dosage.

### Methods of Use

In one embodiment, the anti-NKG2D antibodies disclosed herein can be used to treat diseases such as cancer, autoimmune diseases, GvHd, viral infection or bacterial infection. In another embodiment, the anti-NKG2D antibodies disclosed herein can be used to treat diseases associated with NKG2D. In another embodiment, the anti-NKG2D antibodies disclosed herein can be used to treat diseases associated with over-expression of NKG2D.

In some embodiments, the anti-NKG2D antibodies disclosed herein comprise cytotoxic activities. In some embodiments, the anti-NKG2D antibodies disclosed herein are cytotoxic to cancer or tumor cells.

In some embodiments, the anti-NKG2D antibodies disclosed herein may be used in a method to a cancer or tumor. In some embodiments, the cancer or tumor comprises a solid cancer or tumor. In some embodiments, the cancer or tumor comprises a non-solid (diffuse) cancer or tumor. In some embodiments, the cancer or tumor comprises a metastasis of a cancer or tumor.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The terms “subject,” “individual,” and “patient” are used interchangeably herein, and refer to human or non-human animals to whom treatment with a composition or formulation in accordance with the present anti-NKG2D antibodies is provided. The terms “non-human animals” and “non-human mammals” are used interchangeably herein and include all vertebrates, e.g., mammals, such as non-human primates (e.g. higher primates), sheep, dog, rodent (e.g. mouse or rat), guinea pig, goat, pig, cat, rabbits, cows, horses, or non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In one embodiment, the human is an adult. In another embodiment, the human is a child. The human can be male, female, pregnant, middle-aged, adolescent, or elderly.

Pharmaceutical compositions suitable for use in the methods disclosed herein include compositions wherein the active ingredients are contained in an amount effective to achieve the intended purpose. In one embodiment, a therapeutically effective amount means an amount of active ingredients effective to prevent, alleviate or ameliorate symptoms of disease or prolong the survival of the subject being treated. Determination of a therapeutically effective amount is well within the capability of those skilled in the art.

As used herein, “modulating” refers to “stimulating” or “inhibiting” an activity of a molecular target or pathway. For example, a composition modulates the activity of a molecular target or pathway if it stimulates or inhibits the activity of the molecular target or pathway by at least 10%, by at least about 20%, by at least about 25%, by at least about 30%, by at least about 40%, by at least about 50%, by at least about 60%, by at least about 70%, by at least about 75%, by at least about 80%, by at least about 90%, by at least about 95%, by at least about 98%, or by about 99% or more relative to the activity of the molecular target or pathway under the same conditions but lacking only the presence of the composition. In another example, a composition modulates the activity of a molecular target or pathway if it stimulates or inhibits the activity of the molecular target or pathway by at least 2-fold, at least 5-fold, at least 10-fold, at least 20-fold, at least 50-fold, at least 100-fold relative to the activity of the molecular target or pathway under the same conditions but lacking only the presence of the composition. The activity of a molecular target or pathway may be measured by any reproducible means. The activity of a molecular target or pathway may be measured in vitro or in vivo. For example, the activity of a molecular target or pathway may be measured in vitro or in vivo by an appropriate assay known in the art measuring the activity. Control samples (untreated with the composition) can be assigned a relative activity value of 100%.

In one embodiment, the method comprises the step of administering to the subject a composition comprising an effective amount of the anti-NKG2D antibody disclosed herein. In one embodiment, the composition comprises anti-NKG2D antibodies having the heavy chain and light chain CDR sequences as described herein. In another embodiment, the composition comprises anti-NKG2D antibodies having the VH and VL sequences as described herein.

One skilled in the art would appreciate that in some embodiments, modulation of an immune response encompasses a reduction of inflammation or elimination of inflammation in a situation wherein the expected outcome without the use of an anti-NKG2D antibody described herein, would have been inflammation. One skilled in the art would also appreciate that in some embodiments, treating a tumor or cancer encompasses a reduction of tumor size, growth, and or spread of the tumor or cancer, compared with the outcome without the use of an anti-NKG2D antibody described herein.

In one embodiment, the present disclosure provides a method of treating a disease in a subject, comprising the step of administering to the subject a composition comprising an effective amount of the anti-NKG2D antibody disclosed herein. In one embodiment, the composition comprises anti-NKG2D antibodies having the heavy chain and light chain CDR sequences as described herein. In another embodiment, the composition comprises anti-NKG2D antibodies having the VH and VL sequences as described herein.

In one embodiment, the present disclosure also provides uses of a composition comprising anti-NKG2D antibodies for treating a disease in a subject. In one embodiment, the composition comprises anti-NKG2D antibodies having the heavy chain and light chain CDR sequences as described herein. In another embodiment, the composition comprises anti-NKG2D antibodies having the VH and VL sequences as described herein.

In one embodiment, the exact amount of the present polypeptides or compositions thereof required to elicit the desired effects will vary from subject to subject, depending on the species, age, gender, weight, and general condition of the subject, the particular polypeptides, the route of administration, and whether other drugs are included in the regimen. Thus, it is not possible to specify an exact amount for every composition. However, an appropriate amount can be determined by one of ordinary skill in the art using routine experimentation. Dosages can vary, and the polypeptides can be administered in one or more (e.g., two or more, three or more, four or more, or five or more) doses daily, for one or more days. Guidance in selecting appropriate doses for antibodies can be readily found in the literature.

In one embodiment, the disease can be viral infection, bacterial infection, cancer, autoimmune disease or immune disorder. In one embodiment, the disease can be upper respiratory viral infections, early stage lung infections, or late stage lung infections. A number of diseases and cancer are known to be caused by viruses. Examples of disease-causing viruses include, but are not limited to, norovirus; rotavirus; hepatitis virus A, B, C, D, or E; rabies virus, West Nile virus, enterovirus, echovirus, coxsackievirus, herpes simplex virus (HSV), HSV-2, varicella-zoster virus, mosquito-borne viruses, arbovirus, St. Louis encephalitis virus, California encephalitis virus, lymphocytic choriomeningitis virus, human immunodeficiency virus (HIV), poliovirus, zika virus, rubella virus, cytomegalovirus, human papillomavirus (HPV), enterovirus D68, severe acute respiratory syndrome (SARS) coronavirus, Middle East respiratory syndrome coronavirus, SARS coronavirus 2, Epstein-Barr virus, influenza virus, respiratory syncytial virus, polyoma viruses (such as JC virus, BK virus), Ebola virus, Dengue virus, or any combination thereof.

In another embodiment, the disease is a cancer that can be, but is not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoetic cancer, Non-hodgkin lymphoma, skin cancer, basal-cell carcinoma, melanoma, small cell lung cancer, non-small cell lung cancer, or any combination thereof.

Examples of autoimmune diseases or disorders include, but are not limited to, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including Balanitis circumscripta plasmacellularis, balanoposthitis, erythema annulare centrifugum, erythema dyschromicum perstans, eythema multiform, granuloma annulare, Lichen nitidus, Lichen sclerosus et atrophicus, Lichen simplex chronicus, Lichen spinulosus, Lichen planus, Lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-0 blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes). Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including Polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including Pemphigus vulgaris, Pemphigus foliaceus, pemphigus mucus-membrane pemphigoid, and Pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopeniaurpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCK), acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic reperfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis.

In some embodiments, the disease is a transplantation-related diseases such as graft-versus-host disease (GvHD). According to one embodiment, the GVHD is acute GVHD. According to another embodiment, the GVHD is chronic GVHD.

In another embodiment, the present disclosure provides a method of using a polynucleotide to treat a disease or condition as described above, wherein the polynucleotide encodes an anti-NKG2D antibody as described herein.

As used herein, the terms “comprise”, “comprises”, “comprising”, “includes”, “including”, “having” and their conjugates mean “including but not limited to”.

As used herein, the singular form “a”, “an” and “the” include plural references unless the context clearly dictates otherwise. For example, the term “an antibody” or “at least one antibody” may include a plurality of antibodies.

Throughout this application, various embodiments of the present disclosure may be presented in a range format. It should be understood that the description in range format is merely for convenience and brevity and should not be construed as an inflexible limitation on the scope of the anti-NKG2D antibodies and uses thereof. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When values are expressed as approximations, by use of the antecedent “about,” it is understood that the particular value forms another embodiment. All ranges are inclusive and combinable. In one embodiment, the term “about” refers to a deviance of between 0.1-5% from the indicated number or range of numbers. In another embodiment, the term “about” refers to a deviance of between 1-10% from the indicated number or range of numbers. In another embodiment, the term “about” refers to a deviance of up to 20% from the indicated number or range of numbers. In one embodiment, the term “about” refers to a deviance of +10% from the indicated number or range of numbers. In another embodiment, the term “about” refers to a deviance of +5% from the indicated number or range of numbers.

Unless otherwise defined, all technical and/or scientific terms used herein have the same meaning as commonly understood by one of ordinary skill in the art to which the anti-NKG2D antibodies and uses thereof pertains. Although methods and materials similar or equivalent to those described herein can be used in the practice or testing of embodiments of the anti-NKG2D antibodies and uses thereof, methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of making and using the anti-NKG2D antibodies and variations thereof are described. This description is not intended to be limiting and changes in the components, sequence of steps, and other variations would be understood to be within the scope of the present anti-NKG2D antibodies and uses thereof.

It is appreciated that certain features of the anti-NKG2D antibodies and uses thereof, which are, for clarity, described in the context of separate embodiments, may also be provided in combination in a single embodiment. Conversely, various features of the anti-NKG2D antibodies and uses thereof, which are, for brevity, described in the context of a single embodiment, may also be provided separately or in any suitable subcombination or as suitable in any other described embodiment of the anti-NKG2D antibodies and uses thereof. Certain features described in the context of various embodiments are not to be considered essential features of those embodiments, unless the embodiment is inoperative without those elements.

Various embodiments and aspects of the present anti-NKG2D antibodies as delineated hereinabove and as claimed in the claims section below find experimental support in the following examples.

### EXAMPLES

### Example 1

### Mouse Hybridoma Monoclonal Antibody Generation for NKG2D

Objective: To generate monoclonal antibodies against NKG2D using mouse hybridoma technology.

Methods: Generation of Mouse Hybridoma Anti-NKG2D Antibodies. Anti-NKG2D antibodies were developed by immunizing Balb/c and SJL mice with NKG2D-ECD-hFc (extracellular domain of NKG2D fused to human Fc). The animals were bled and tested for antibody titer by ELISA (test-bleed 1 and test-bleed −2). Spleen cells from immunized mice with high titer were isolated and fused by standard fusion procedures to create hybridoma producing specific antibodies. Supernatants containing antibodies produced by pools of these cells were primary screened by ELISA for reactivity with NKG2D-ECD protein fused to human Fc (NKG2D-ECD-hFc) and a secondary screening by FACS for reactivity with NKG2D over-expressing cells. For screening, large numbers of hybridoma supernatants for target antibody activity by FACS were tested.

The target over-expressing cells were placed into 96-well round-bottom polystyrene plates and incubated with neat supernatants from the hybridoma cultures. The cells were then washed, incubated with a fluorescence labeled secondary antibody. As a negative reference, non-target protein gene transfected parental cells were employed to test the supernatants (and found to be negative) to confirm that the reactive antibody recognized target protein specifically. Positive pools were identified and cloned by limiting dilution. After 1-2 fusions, positive clones producing specific antibodies were identified and selected by ELISA and FACS.

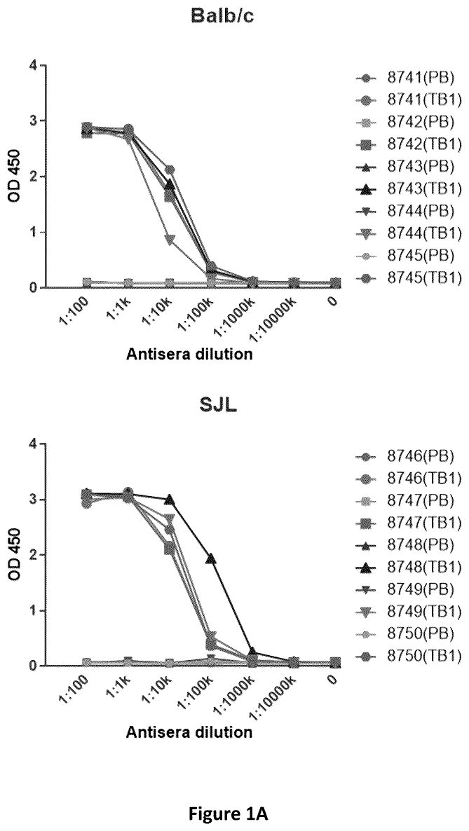

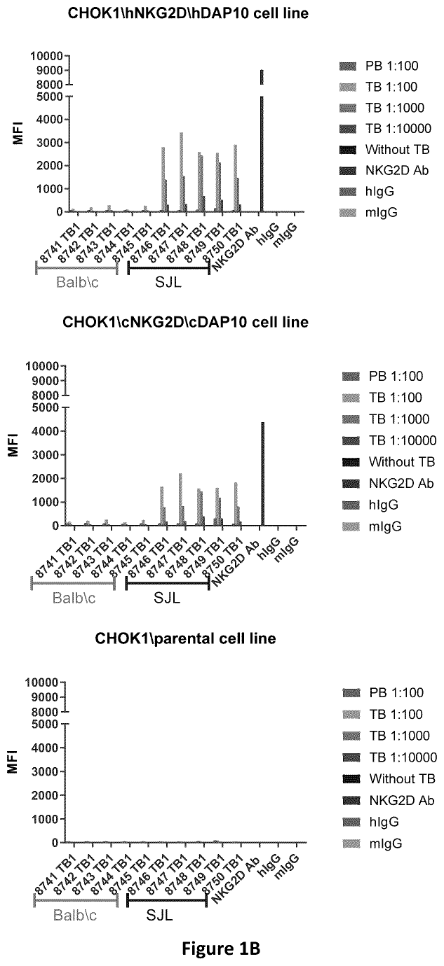

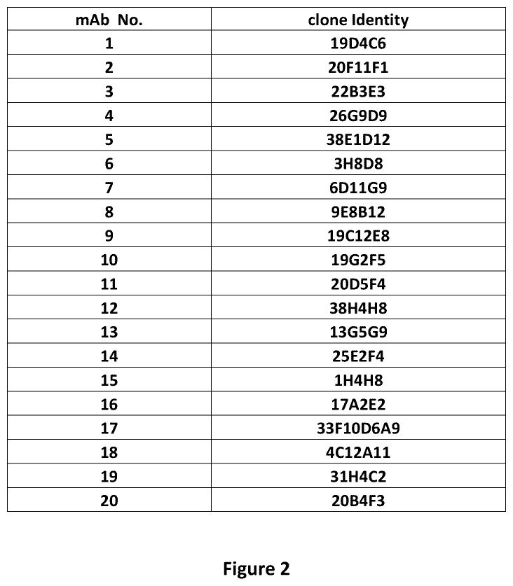

Results: Following animals' immunization, serum was tested for binding to recombinant human NKG2D-ECD-Fc (extracellular domain of NKG2D fused to human Fc) by ELISA and by FACS (FIG. 1). As shown in FIG. 1A test bleed (TB) 1 of Balb/c animals or SJL animals—in various dilutions showed positive titer against the human NKG2D-ECD-Fc by ELISA as compared to the pre-bleed (PB) samples. FIG. 1B demonstrate serial dilutions of test bleed 1 titer against the human-NKG2D/Dap10 over expressed in CHO cells, and against the cyno-NKG2D/Dap10 over expressed in CHO cells, while no reactivity on CHO parental cells. SJL mice showed stronger immune response as compared to Balb/c mice when tested for binding to NKG2D/Dap10 CHO cells. Following test-bleed 2 with similar observations (data not shown), animals SJL #8748 and #8749 were selected for fusion. Primary screen was obtained following electrofusion to identify positive clones by ELISA against recombinant human NKG2D-ECD-Fc, with human Fc served as a negative control. Secondary screen was obtained against CHO cells over expressing human-NKG2D/Dap10 or cyno-NKG2D/Dap10 to identify cross reactive clones, with CHO parental cells served as negative control. In the primary screen, 103 clones were selected for secondary screening. Secondary screen revealed clones that were further subcloned, and finally, 18 clones were proceeded for 200 ml expression and protein A purification. FIG. 2 presents the identity of the selected clones.

Summary: Mouse monoclonal hybridoma generation against human NKG2D protein, yielded 20 clones that were identified following primary and secondary screening by ELISA and FACS to be specific binders to human and cyno NKG2D. These clones were further expressed purified, analyzed, and characterized for binding efficacy.

### Example 2

### Binding Characterization of the Mouse Hybridoma Monoclonal Antibodies Against Human-NKG2D

Objective: To express purify and characterize mouse monoclonal Abs against NKG2D by binding.

Methods: Expression and Purification of the Hybridoma Clones. In Brief, ˜0.25-0.5×10⁷ cells were inoculated into a roller bottle pre-filled with 100 mL antibody production medium (Hybridoma-SFM+2.5% FBS (Low IgG), and incubated in roller culture apparatus at 300 r/h speed for 14-16 days at 37° C., no CO2 condition. Thereafter, the cell suspension was transferred into a 350 mL centrifuge bottle and centrifuged at 3,220 g, 4° C., for 15 min, and then filtered with a 0.45 μm filtration capsule to remove the cells and cell debris. Then the culture supernatant was loaded onto a pre-equilibrated Protein A affinity column for affinity purification. Antibody was eluted from the column with 5 CV (Column Volume) of elution buffer (0.1 M citrate sodium buffer, pH 3.0), and neutralized to final pH 7.0 with Trizma base, and then dialyzed against 100-fold of elution volume of PBS, pH 7.4, at 2-8° C. overnight, and sterile-filtered with a 0.22 μm syringe filter in a biological safety cabinet. The purified antibody was then aliquoted and stored at −20° ° C. or −80° C. until use.

ELISA Binding to the Target Protein. Briefly, dilute target protein NKG2D-ECD-Fc into PBS with final concentration of 0.7 μg/mL, and coat 100 μL/well on ELISA plate (cat: 9018, supplier Corning). Incubate O/N, 4° C. The plates were blocked with 250 μL 1% BSA in PBST for 1 hr at 37° C., washed four times with PBST using Biotek (Elx 405). All the Abs were serial diluted and 100 μL/well diluted antibody were added to plate, incubate for 1 hr at 37° C., wash 4 times with PBST. After 100 μL/well goat Anti-Mouse IgG (Fab specific)-HRP (SIGMA A3682) 1:10000 were added, cells were incubated for 0.5 hr at 37° C. Following 4 times washing with PBST, 100 μL/well of TMB substrate was added and incubated at room temperature for 5 min. 100 L/well of 1N HCl to terminate reaction were added. Plates were read using ELISA plate reader at 450 nm wavelength (instrument SpectraMax M5e). Data Analysis was performed using Graphpad prism 5 software by using nonlinear regression (curve fit): log (agonist) vs. response, agonist is antibody concentration (nM) and response is OD value.

FACS Binding to Cells. In Brief, directly harvest suspension cultured cells or TrypLE Express Enzyme (cat: 12604-013, supplier Life technologies) digest adherent cells before harvesting. Centrifuging at 1000 rpm for 5 min and discard the supernatant. Cells are suspended at a concentration of 2×10⁶/mL in FACS buffer (2% FBS in PBS) and 100 μL/well of cell suspension added to the plate (cat #3799, supplier Corning). Plates were Centrifuged at 2000 rpm for 5 min, and the supernatant was discarded. Cells were then re-suspended in 100 μL/well of Tribody set antibodies (400 nM start, 4-fold dilution, 8 point including 0 point) and plate was incubated for 60 min at 4° C. Plates were centrifuged at 2000 rpm, 4° C. for 5 min and supernatant were discarded. Then cells were washed 3 times with 170 μL FACS buffer, re-suspended at 100 L/well with secondary antibody and incubated for 30 min at 4° C. in dark. Plates were centrifuged at 2000 rpm, 4° C. for 5 min and supernatant was discarded. Then cells were washed 3 times with FACS buffer and analyzed with FACS verse.

Epitope Binning by ELISA. Briefly, dilute target antibodies into PBS with final concentration of 1 μg/ml and coat 100 μL/well on ELISA plate (cat: 9018, supplier Corning). Incubate O/N, 4° C. After blocking with 250 μL 1% BSA in PBST for 1 hr at 37° C., add series concentration of biotinylated antigen hNKG2D-ECD-Fc. Wash the plate 3 times with PBST after incubating for 1.5 hrs at 37° ° C., then add 100 L of streptavidin-HRP (cat: S5512, supplier Sigma, 1:10000) to each well. After incubating for 1 hr at 37° C., wash the plate 4 times with PBST. 100 μL/well of TMB substrate was added and incubated at room temperature for 5 min. 100 μL/well of 1N HCL to terminate reaction. Plates were read using ELISA plate reader at 450 nm wavelength. Find EC80 of antigen using Graphpad prism 5 software.

The following describes the setup of serial dilutions of mAbs that were mixed with the protein before binding to the mAb coated plate. Dilute target antibodies of anti-human NKG2D into PBS with final concentration of 1 μg/mL and coat 100 μL/well on ELISA plate (cat: 9018, supplier Corning). Incubate O/N, 4° C. After blocking with 250 μL 1% BSA in PBST for 1 hr at 37° ° C., add mixture of biotinylated antigen at EC80 (2-fold preparation, add 50 μL) and competitive antibodies (40-80 fold preparation, add 50 μL). Wash the plate 3 times with PBST after incubating for 1.5 hrs at 37° C., then add 100 μL of streptavidin-HRP (cat: S5512, supplier Sigma, 1:10000) to each well. After incubating for 1 hr at 37° C., wash the plate 4 times with PBST. 100 μL/well of TMB substrate was added and incubated at room temperature for 5 min. 100 μL/well of 1N HCl to terminate reaction. Plates were read using ELISA plate reader at 450 nm wavelength.

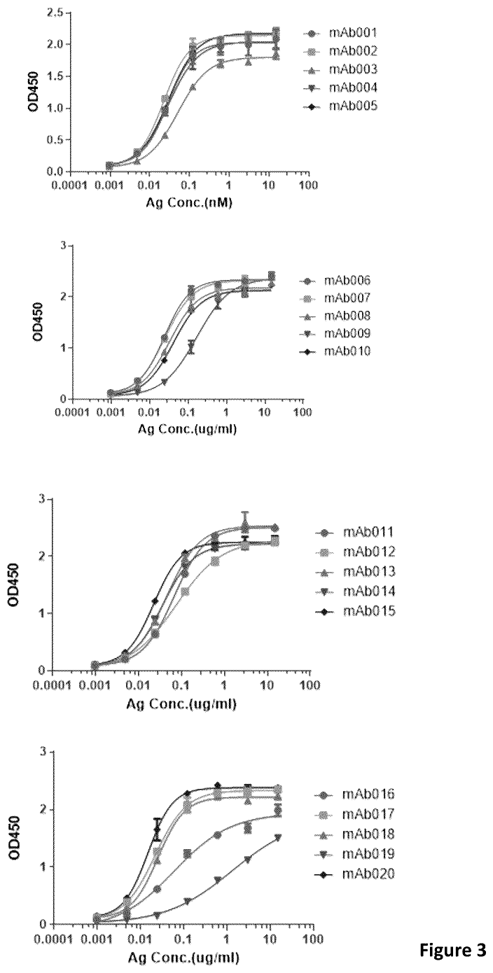

Results: FIG. 3 present the binding of the purified mAbs to the NKG2D-ECD-Fc antigen by ELISA. Excluding mAb 019, EC80 values range of 0.03 nM to 0.6 nM among these antibodies were observed.

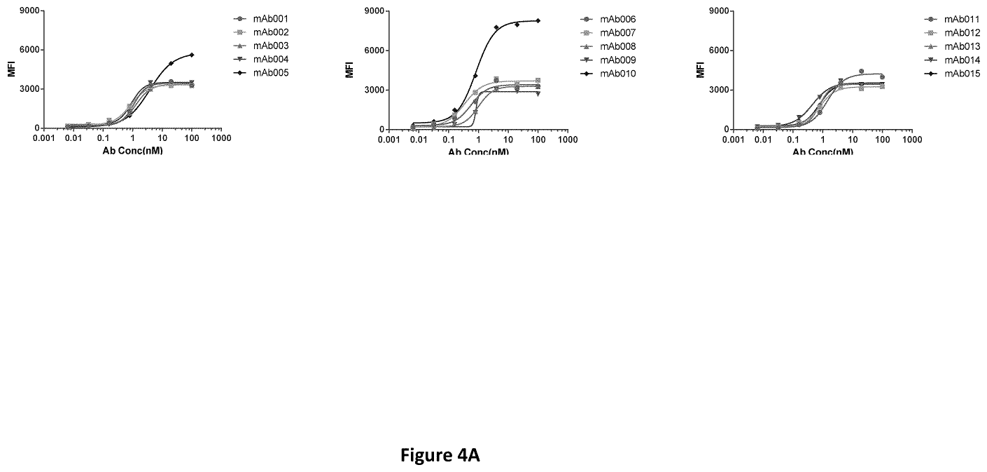

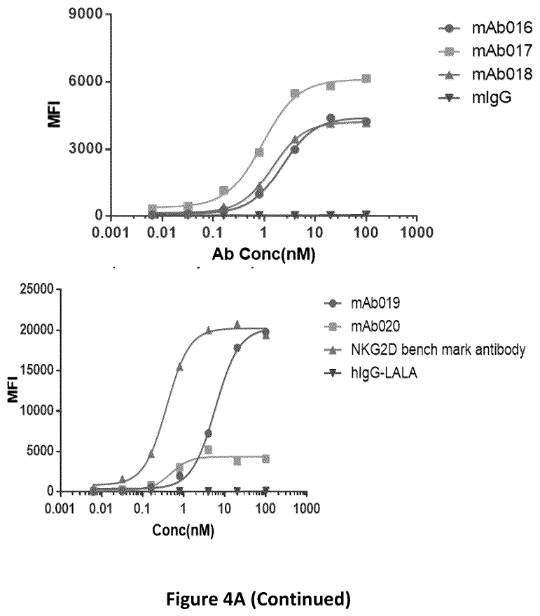

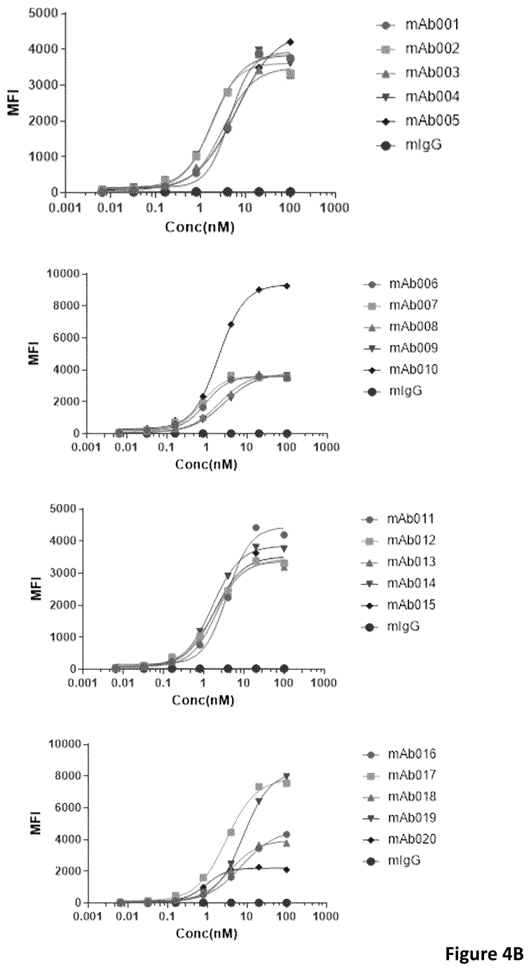

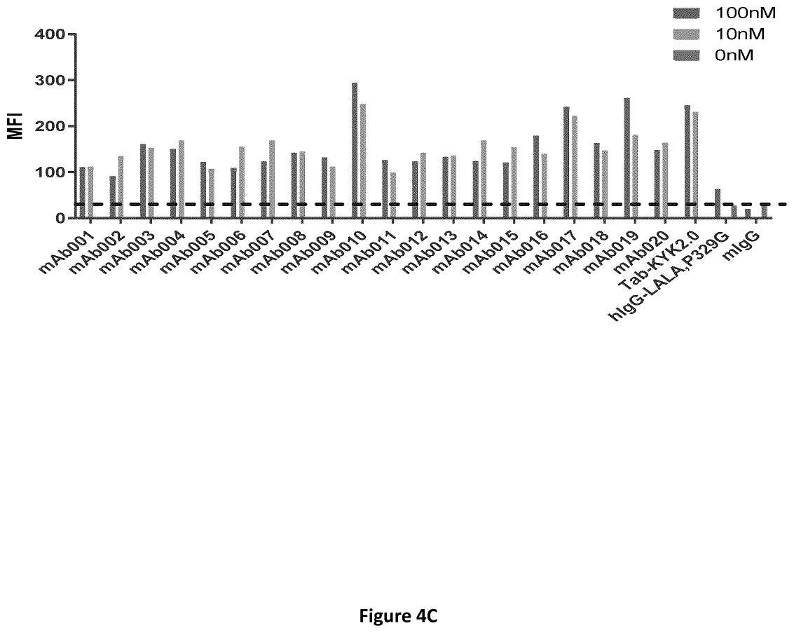

FIG. 4 present the FACS binding of selected purified mAbs to the CHO over expressing human/cyno NKG2D/Dap10, as well as to primary NK cells purified from healthy donor's PBMCs. As shown in FIG. 4, all purified mAbs showed binding activity on CHOK1-hNKG2D, CHOK1-cNKG2D and primary NK cells with various range of affinity and efficacy.

EC50 value range of ˜0.4-3.8 nM binding to the CHO cells over expressing the human NKG2D/Dap10 were observed (FIG. 4A). Similarly, EC50 value range of ˜0.7-8 nM binding to the CHO cells over expressing the cynoNKG2D/Dap10 were observed (FIG. 4B). Two concentrations tested for binding to primary NK cells, showed dose dependent binding in all mAbs with various MFI (FIG. 4C). No binding is observed on CHO parental cells (data not shown).

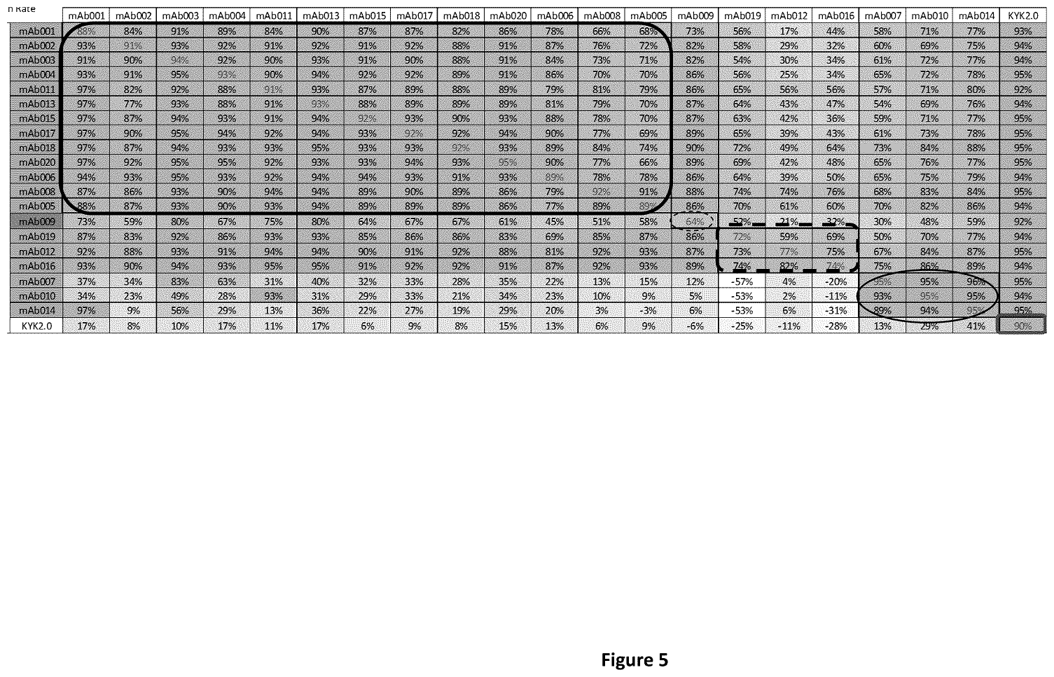

FIG. 5 summarize the epitope binning analysis performed by ELISA. ELISA analysis suggests 4 groups differentiated in their epitope bin as follows: Group1 (square continuous line) gather mAb001, 002, 003, 004, 005, 006, 008, 011, 013, 015, 017, 018, 020. Group 2 (circle continuous line) gather mAb007, 010, 014. Group 3 (square dashed line) gather mAb012, 016, 019. Group4 (circle dashed line) gather mAb009. The positive control Ab differ from all groups.

Summary: Monoclonal antibodies (mAbs) against human NKG2D were successfully generated using hybridoma technology. 20 clones were identified and characterized. Selected mAbs were further expressed and produced by the hybridoma clones. mAbs were further purified, analyzed by MS (Mass-Spec) and characterized for ELISA binding to NKG2D-ECD-Fc antigen, as well as FACS binding to cells expressing human and cyno NKG2D/Dap10, and finally tested for epitope binning by ELISA, which yielded 4 bin groups.

### Example 3

### In Vitro Functional Evaluation of the Mouse Hybridoma Monoclonal Antibodies

Objective: To evaluate in vitro NK cell activation upon anti NKG2D mAbs binding to primary NK cells, by CD107a and IFNg upregulation.

Methods: Briefly, Isolate NK cells from PBMCs collected from healthy donor using EasySep™ Human NK Cell Isolation Kit (STEMCELL, Cat-17955). Suspend NK cells with RPMI 1640 (Gibco, Cat-A10491-01) containing 10% FBS and 100 ng/mL hIL-2 (R&D SYSTEMS, cat-219-IL) for 2 days. Crosslink the antibody by coating them onto 96-well plate (Cat-3599, Corning) and then incubate overnight at 4° C. Remove supernatant from the coated plate and then wash the plate twice with 200 μL PBS. Harvest stimulated NK cells by centrifuging at 500 g for 5 min, adjust the concentration of cells to 5×10⁵/mL with NK cell stimulation medium (RPMI 1640+10% FBS+20 ng/mL hIL-2), then add 200 μL/well NK cells to the Ab coated plate. Add APC anti-CD107a (Biolegend, cat: 328620) and control (0.5 μL/test) respectively into NK cells separately. Incubate the plate in a humidified 5% CO₂ atmosphere at 37° C. for 4 hrs. Collect supernatant for IFN-gamma measurement and cells for CD107a measurement, respectively. CD107a measurement: Transfer cells to FACS plate (Cat-3799, Corning). Wash the cells twice with 200 μL FACS buffer (2% FBS in PBS). Add APC-anti-CD107a, FITC-anti-CD3 (Invitrogen, cat: 17-0037-41) and BV421-anti-CD56 (BioLegend, cat: 318328) and control respectively into NK cells suspension and incubate cells for 1 hr at 4° C. After 2× washing with 200 μL FACS buffer, re-suspend the cells in 100 μL cold PBS. Keep the cells in dark and submit the cells for FACS (FACS Canto II, BD Biosciences) analysis. IFN-gamma measurement: the IFN-gamma was measured according to the protocol of Human INF gamma DuoSet ELISA kit (R&D, DY285). Briefly, coat the Capture Antibody at working concentration in PBS onto a 96-well microplate (Cat-9018, Corning), incubate overnight at RT. After 3× wash, block plates with 300 μL of Reagent Diluent (1% BSA in PBS) for at least 1 hr at RT. After 3× of wash, add 100 μL sample with proper dilution or standards in Reagent Diluent, and incubate for 2 hrs at RT. After the three times of wash, add 100 μL diluted Detection Antibody, and incubate for 2 hrs at RT. After the three times of wash, add 100 μL of the working dilution of Streptavidin-HRP B, and incubate for 20 min at RT. After the three times of wash, add 100 μL of Substrate Solution, and incubate for 20 min at RT. Add 50 μL of Stop Solution. Read the OD450 using a microplate reader (Molecular Device, cat: Spectra Max M5e).

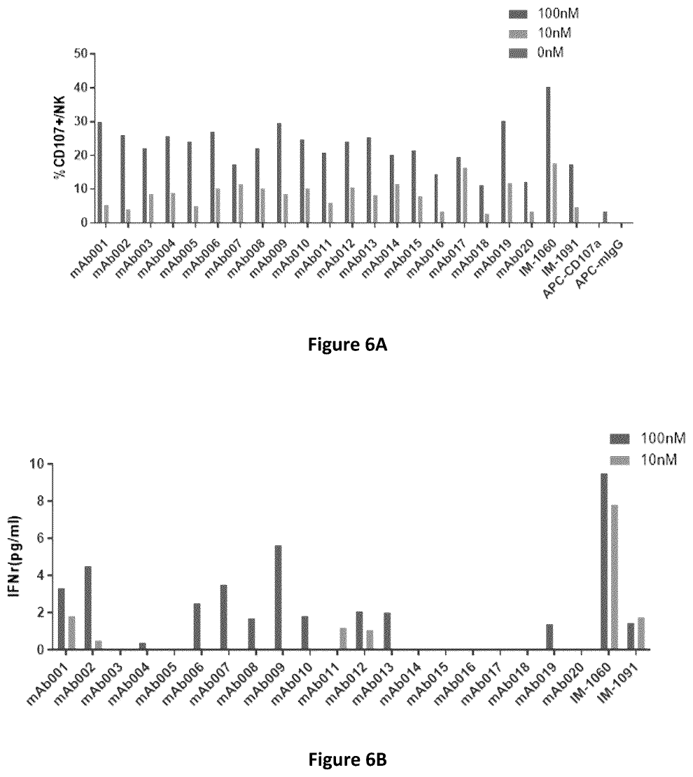

Results: FIG. 6 present the activation of NK cells upon binding of the purified anti NKG2D mAbs (at 10 nM or 100 nM), as determined by accumulation of CD107a activation marker or by IFN-gamma secretion. Assay validity was confirmed by a window determined by IM-1060 positive control Tribody and IM-1091 negative control Tribody. Dose dependent CD107a⁺NK cells accumulation was determined upon mAbs binding by FACS, as compared to mouse IgG control (FIG. 6A). 10 out of the tested mAbs also stimulated IFN-gamma secretion measured in the supernatants by ELISA (FIG. 6B).

Summary: Twenty purified anti-NKG2D mAbs were characterized by binding and activation. The Abs were ranked based on their binding activity as well as their NK activation activity. Four mAbs (mAb 001, mAb009, mAb010 and mAb019), one from each bin, were confirmed by Mass-Spec (data not shown) and further selected based on their characteristics exhibited, for introducing to a Tribody construct and evaluation.

### Example 4

### In Vitro Evaluation of Blocking NKG2D-MICA Interaction by the Mouse Hybridoma Monoclonal Antibodies

Objective: To evaluate in vitro the blocking activity of the purified mAbs on receptor-ligand NKG2D-MICA interaction.

Methods: Competitive ELISA Based RBA: Dilute target protein (NKG2D-ECD-hFc, Lot: 20200413002, CP) into PBS with final concentration of 1 μg/ml and coat 100 μL/well on ELISA plate (cat: 9018, supplier Corning). Incubate O/N, 4° C. After blocking with 250 μL 1% BSA in PBST for 1 hr at 37° C., add mixture of series concentration of Mabs diluted 1:5 from 300 nM (600 nM preparation, add 50 μL) and Human MICA-His at EC₈₀ (2-fold preparation, add 50 μL). Wash the plate 3 times with PBST after incubating for 1.5 hrs at 37° C., then add 100 μL of anti-His-HRP (1:5000) to each well. After incubating for 1 hr at 37° C., wash the plate 4 times with PBST. 100 μL/well of TMB substrate was added and incubated at room temperature for 5 min. 100 L/well of 1N HCl to terminate reaction. Plates were read using ELISA plate reader at 450 nm wavelength.

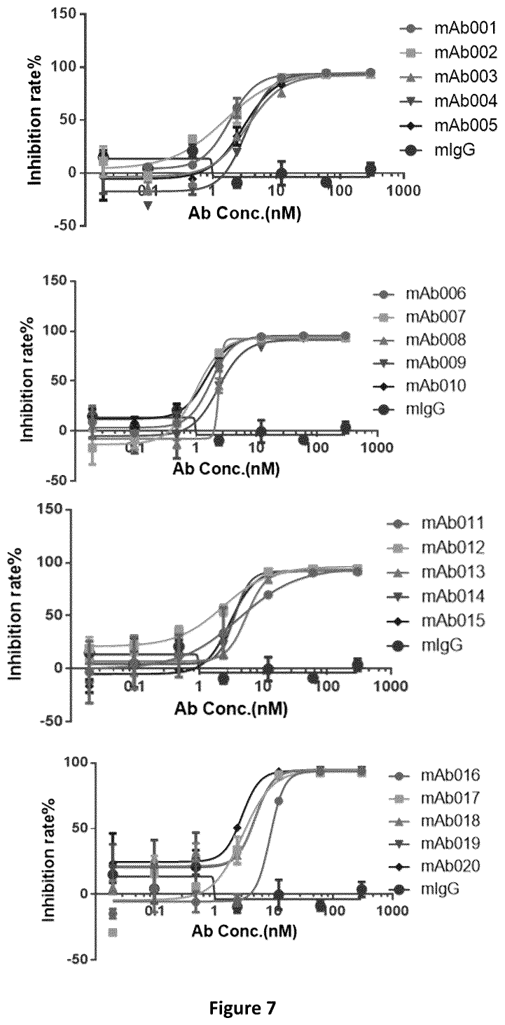

Results: mAbs were analyzed for their ability to block NKG2D-MICA interaction. As shown in FIG. 7, the range of the inhibition rate demonstrated as IC50 values was 1.4 nM-8 nM, while no inhibition activity was observed in the mIgG1 negative control.

Conclusion: All mAbs tested exhibited blocking activity to the NKG2A-MICA receptor-ligand interaction.

### Example 5

Recombinant Production of Chimeric Tribody with Anti Human NKG2D Derived from the Mouse Hybridoma Clones Against Human-NKG2D

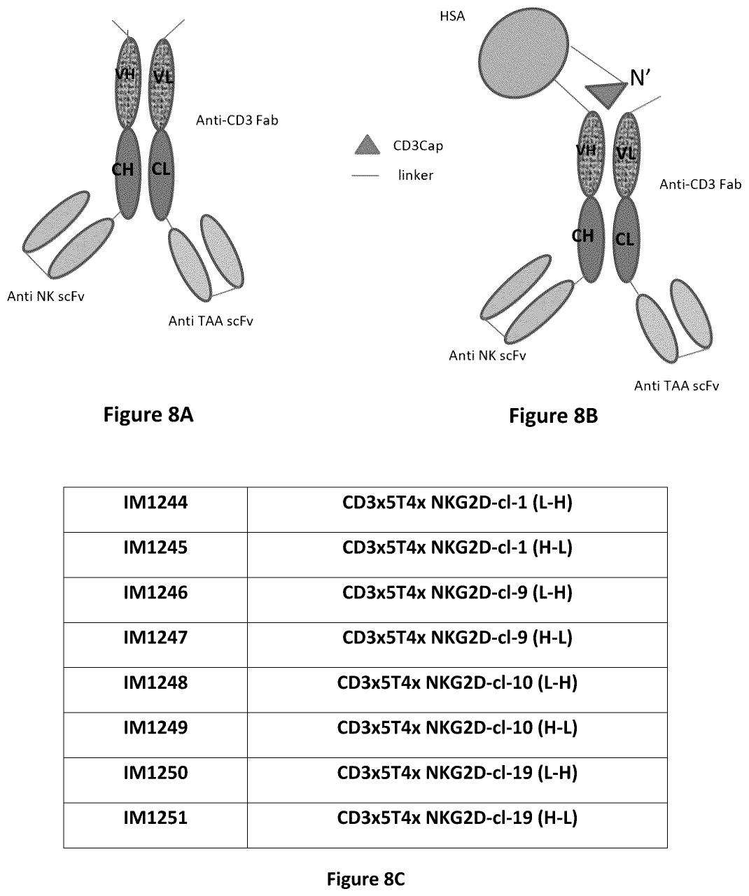

Objective: Introduce the mouse CDRs sequences of the selected anti-human NKG2D mAbs (mAb 001, mAb009, mAb010 and mAb019) to the Tribody/ProTribody constructs, to produce a trispecific chimeric Ab that comprise of anti CD3 Fab, a single chain of anti NKG2D mAb, and a single chain of anti 5T4, as illustrated in FIG. 8A-B respectively, and previously described in WO 2020/225805, incorporated herein in full.

Methods: Hybridoma Sequencing. Briefly, selected positive monoclonal hybridoma cells (˜1×10⁷) were collected for total RNA isolation following the protocol of NucleoZOL Reagent (MACHEREY-NAGEL, 740404.200). Total RNAs were used for cDNA synthesis following the kit manual of SMARTer® RACE 5′/3′, and random primer was used for the syntheses of first-strand cDNA. To amplify the heavy and light chain variable regions with PCR, synthetic cDNA was employed as template, the primers from mouse Ig-Primer Set (Novagen, 69831-3) as Gene-Specific Primer (GSP). PCR products with correct size were collected and purified with NucleoSpin® Gel and PCR Clean-up (Macherey-Nagel, 740609.250) following the Kit's manual, and subjected to TA cloning and sequencing. The heavy chain and the light chain variable regions (VH and VL) of NKG2D-mAbs were cloned into Tribody constructs as describe below, which were then characterized for binding to NKG2D by ELISA and FACS.

Gene Synthesis And Plasmid Construction. The coding sequences for the heavy chain (HC) and light chain (LC) of the trispecific antibody were generated by DNA synthesis and PCR, subsequently subcloned into pCDNA3.4-based plasmid (Invitrogen) for protein expression in mammalian cell system. Finally, the gene sequences in the expression vectors were confirmed by DNA sequencing.

Expression of Trispecific Antibody Construct. Transient expression of the Tribody/ProTribody antibodies was performed by co-transfection of paired HC and LC constructs (at 1:1 HC/LC ratio for the Tribody format or 2.5:1 HC/LC ratio for the ProTribody format) into CHO cells using PEI method. Briefly, 1 L of CHO cells at approximately 5.5×10⁶/ml in a 3 L shake flask was used as the host, Transfection was initiated by adding a mixture of 1 mg of total DNA and 4 mg PEI in 100 ml OptiMEM medium (Invitrogen) to the cells and gentle mixing. Cells were then cultured in an incubator shaker at 120 rpm, 37° ° C., and 8% CO2, for 8-10 days. Feeding with peptone and glucose was carried out 24 h later and every 2-3 days thereafter depending on the cell density and viability. The cell culture was terminated on day 8-10 when cell viability reduced to <80%. The conditioned medium was harvested for protein purification.

Purification of Trispecific Antibody Construct. Protein purification by affinity chromatography and SEC was performed using an AKTA pure instrument (GE Lifesciences). Affinity capture of the Tribody was achieved by passing the harvested supernatants over a column of CaptureSelect™ CH1-XL Affinity Matrix (Thermo Scientific). After washing column with Buffer A (25 mM Tris, 150 mM NaCl, 5 mM EDTA, pH 7.5), the protein was eluted with Buffer B (50 mM Sodium citrate, 150 mM NaCl, pH 3.0), and immediately neutralized with ⅙ volume of Buffer D (1 M Aarginine, 400 mM Succinic acid, pH 9.0). The affinity purified protein was then concentrated to 5-10 mg/ml using Amicon 30 kD concentrator (Merck Millipore) and subjected to SEC purification on a Superdex200 column (GE Lifesciences) equilibrated with SEC Buffer: 200 mM Arginine, 137 mM Succinic acid, 0.05% Tween-80,150 mM NaCl, pH5.0. The target Tribody fractions were collected, then added 5% trehalose (146 mM). The target Tribodies were analyzed using SDS-PAGE and HPLC-SEC.

SDS-PAGE Analysis of Trispecific Antibody Construct. SDS-PAGE analysis of tribody was carried out under reducing and non-reducing conditions in pre-cast polyacrylamide gels. Briefly, 2 ug Tribody samples were mixed by NuPAGE™ LDS sample buffer (thermofisher-NP0008) with 70 mM DTT add or not. After incubating at 25° C. or 90° C. for 10 min, the samples and Unstained Protein Standards (BIO RAD-161-0363 were loaded onto the gels. Electrophoresis was carried out at a constant voltage of 120 V with 1× Tris-glycine-SDS running buffer. Following electrophoresis, gels were stained for overnight using Coomassie blue and de-stained with destaining solution (10% acetic acid, 40% methanol and 50% water). Destained gels were scanned with a Gel imaging system (Tanon-2500R).

SEC-HPLC Analysis of Trispecific Antibody Construct. Analytical SEC-HPLC was performed using Shimadzu LC-10 HPLC instrument (Shimadzu Corp.). 20 μl sample on 1 mg/ml will be loaded to a Superdex 200 Increase 5/150GL column (GE Lifesciences). The mobile phase was 2*PBS with a flow rate of 0.3 ml/min, 15 min.

LC-MS Analysis of Tribody Construct. The Tribody was separated with ACQUITY UPLC BEH200 Å, SEC column (Waters 1.7 μm, 4.6×300 mm) at room temperature and detected by ESI-MS(Thermo, MS-B20-03). The mobile phase was 0.1% formic acid:acetonitrile (75:25, v/v) with a flow rate of 0.2 mL/min. Mass spectrometry was performed in the positive ion. Other parameters for mass spectrometry were: resolution of 17500, Scan range of 1000-5000 m/z, In-source CID of 60 eV, sheath gas flow rate of 30 L/min, capillary temperature of 350° C., Spray voltage of 2.5 KV.

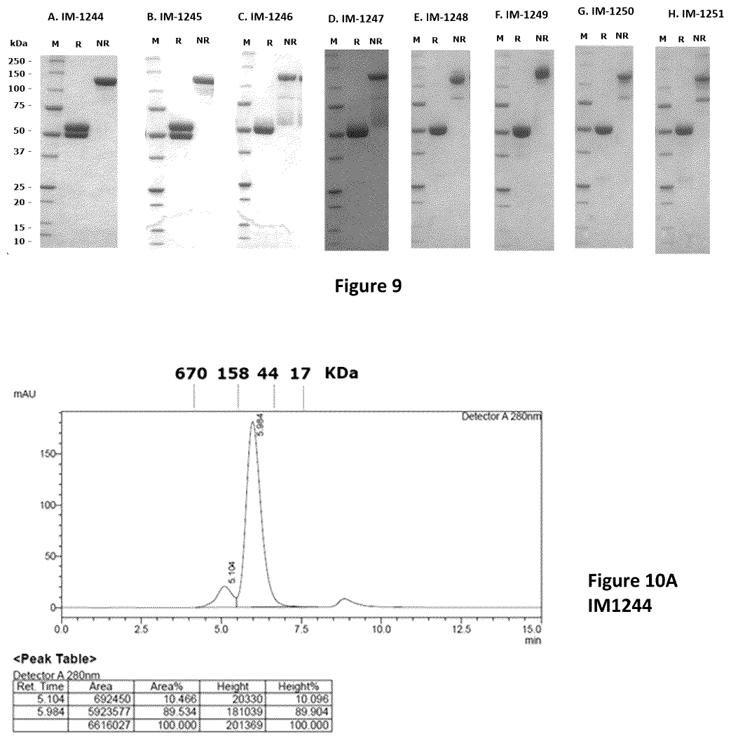

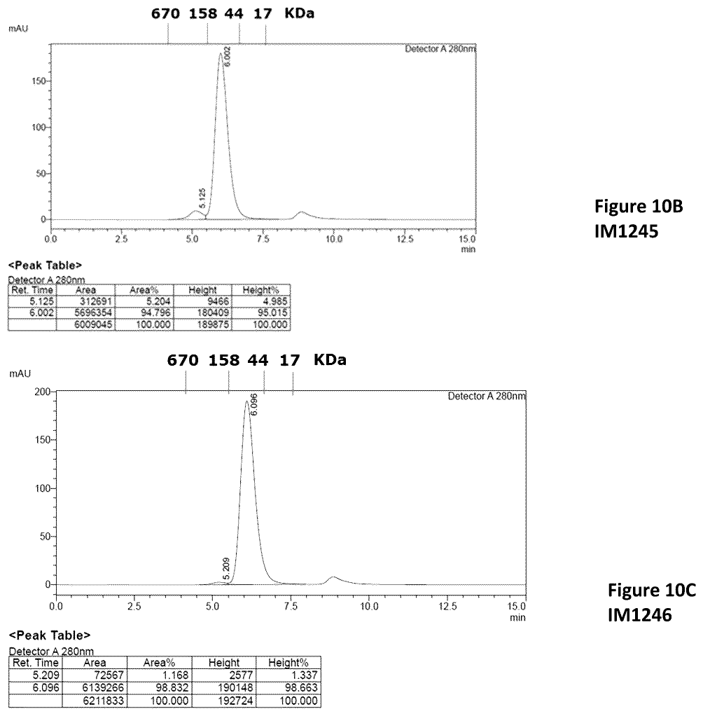

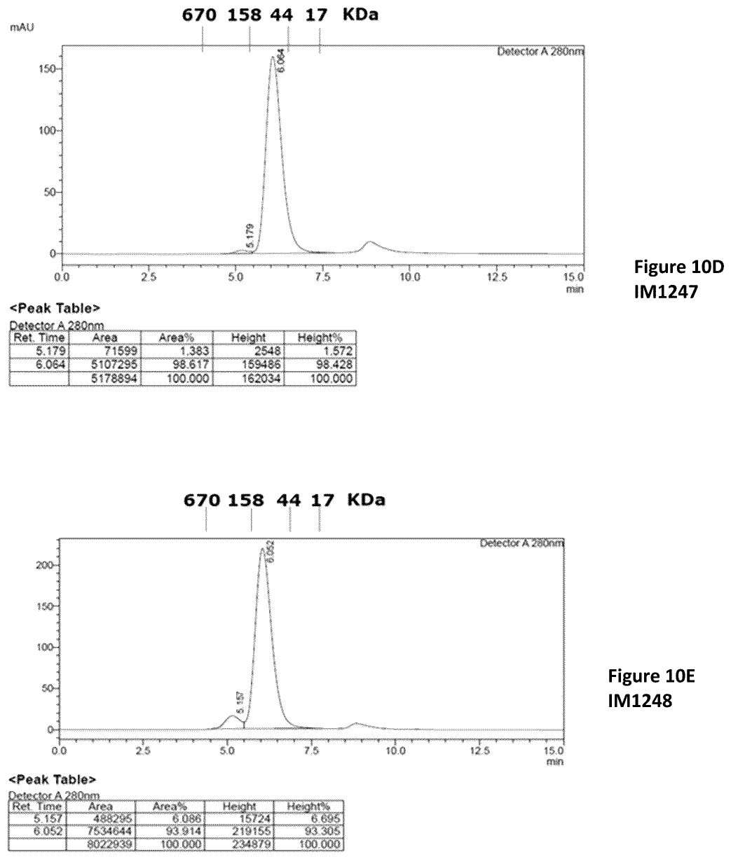

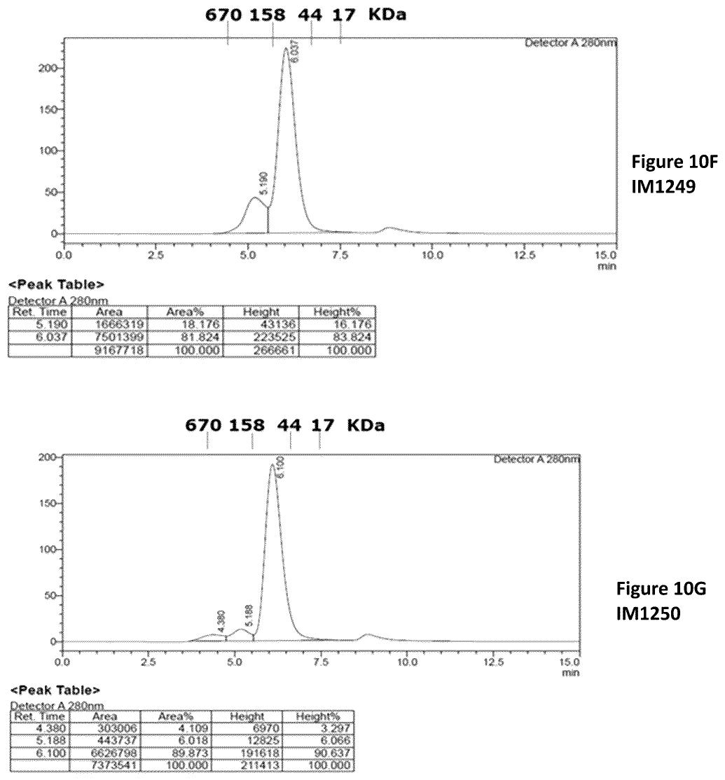

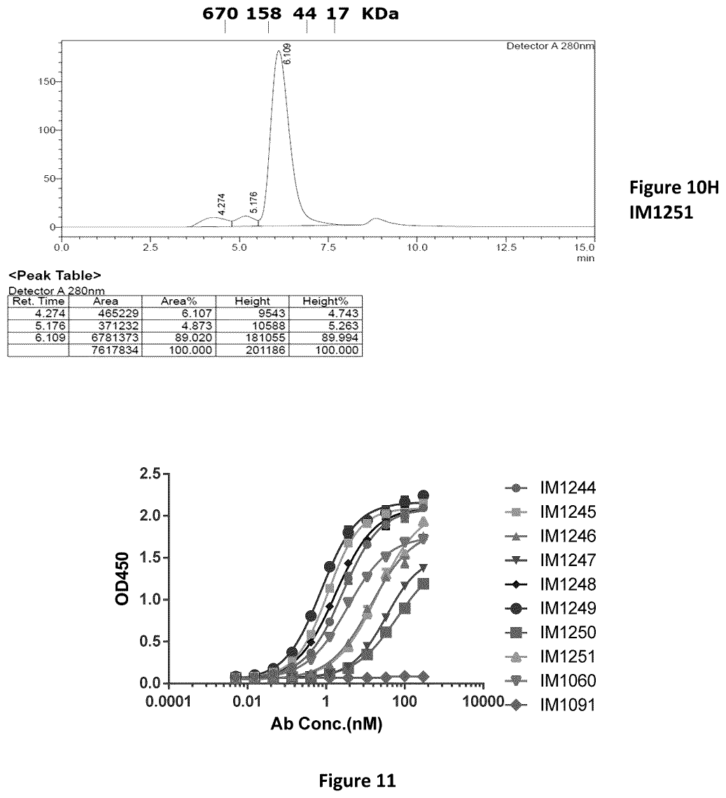

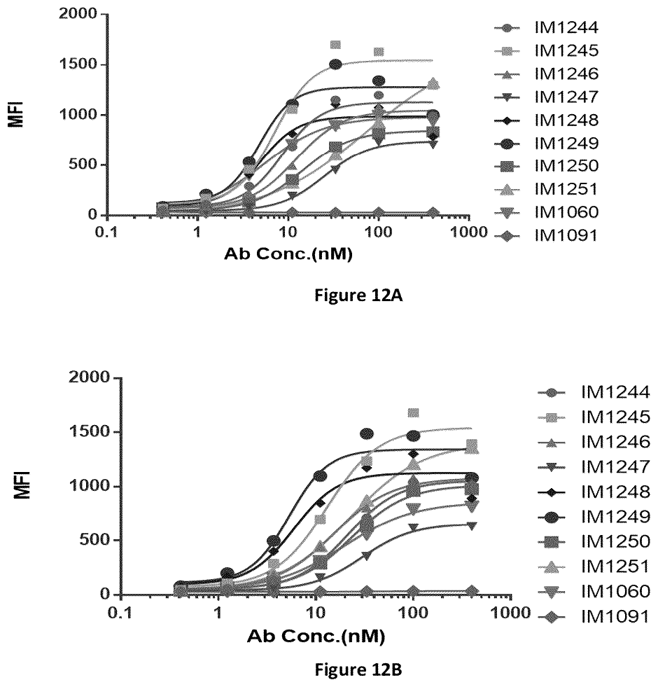

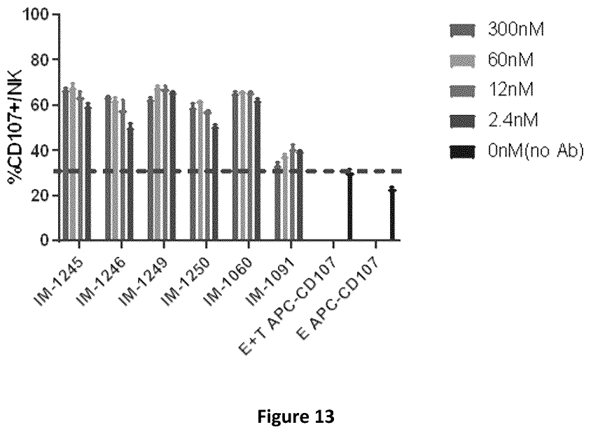

Results: Eight Tribody constructs that present two variants of each of the mAb clone selected (mAb001 represented by IM-1244/5, mAb009 represented by IM-1246/7, mAb010 represented by IM-1248/9 and mAb019 represented by IM-1250/1), were designed to produce a trispecific chimeric Ab that comprise of anti CD3 Fab, a single chain of anti NKG2D, and a single chain of anti 5T4, as illustrated in FIG. 8 either without (FIG. 8A) or with (FIG. 8B) the regulatory domains. FIG. 8C details the Abs identities produced, where L-H indicates for anti NKG2D variable light chain fused upstream to anti NKG2D variable heavy chain, and H-L indicates for anti NKG2D variable light chain fused downstream to anti NKG2D variable heavy chain. The expressed HC and LC Tribody constructs of each of the Tribody molecules associate to form a single molecule, indicated by the single major ˜100 kDA band observed in the SDS-PAGE in non-reduced (NR) conditions, and a double band at ˜50 kDa in reduced (R) conditions (FIG. 9). FIG. 9 presents the SDA-PAGE analysis of IM-1244-IM1251, respectively, where the left lane represents the marker in kDa (M), the middle lane represents the reduced conditions (R), and the right lane represent the non-reduced conditions (NR). FIGS. 10A-H present the SEC-HPLC analysis of IM-1244-IM1251, respectively, and demonstrate a single peak at retention time of ˜6 min, in agreement with the expected retention time of the expected Mw based on mass calibration curve.

Conclusion: A Trispecific Tribody variants were successfully expressed and purified.

### Example 6

### Binding of Tribody Antibody Constructs to NKG2D Recombinant Protein

Objective: To study the binding efficacy of the variety of Tribody antibody constructs that comprised of anti 5T4ScFv domain, anti CD38 Fab domain, and anti humanNKG2D derived from the mouse hybridoma clones, to NKG2D recombinant protein by ELISA and to cells expressing membrane bound NKG2D protein by FACS. The various formats may be comprised of a CAP masking sequences, a cleavable linker, a non-cleavable linker, as well as a point-mutated engager sequences that are lack of binding activity to the specific engager and serve as negative controls for the Tribody/Protribody formats.

Methods: ELISA and FACS binding of Tribody antibody constructs to antigens or cells as describes in Example 2 above.

Results: FIG. 11 demonstrate the expressed trispecific constructs analysis for their binding to NKG2D-ECD-Fc protein by ELISA. The binding EC50 to NKG2D-ECD-Fc protein was 2.6 nM for the IM-1244 (small circles), 1.1 nM for IM-1245 (small squares), 1.7 nM for IM-1248 (small rhombus), 0.7 nM for IM-1249 (big circle), while no binding observed for the negative control Tribody IM-1091 NKG2D mutant variant (big rhombus), as expected (FIG. 11).

FIG. 12 demonstrate the expressed trispecific constructs analysis for their binding to cells expressing NKG2D-protein by FACS. The binding EC50 to CHO cells over expressing human NKG2D were 8.6, 7, 12, 2.5, 4.9, 4.7 nM for IM-1244 (small circle), IM-1245 (small square), IM-1246 (small triangle up), IM-1247 (small triangle down), IM-1248 (small rhombus), IM-1249 (big circle) and IM-1250 (big square), respectively (FIG. 12A). The binding EC50 to CHO cells over expressing cynoNKG2D were 21, 13, 15 30, 5.9, 5.3, and 2.3 nM for IM-1244 (small circle), IM-1245 (small square), IM-1246 (small triangle up), IM-1247 (small triangle down), IM-1248 (small rhombus), IM-1249 (big circle) and IM-1250 (big square), respectively (FIG. 12B). IM-1251 EC50 value (big triangle up) was not applicable, while no binding observed for the negative control Tribody IM-1091 NKG2D mutant variant (big rhombus), as expected. IM-1060 (big triangle down) was used as a positive control Tribody. Binding to CHO parental cells was not detectable (data not shown).

Conclusion: The mouse Variable Light Chain and Heavy Chain sequenced from the selected hybridoma clones were identified and were converted into scFvs and introduced to Tribody constructs to form chimeric TriBody molecules. These molecules were expressed, purified, and further characterized by binding assays and showed that the binding characteristics to human NKG2D was maintained.

As shown in FIG. 11, the binding of the chimeric Tribody that comprised of the anti NKG2D within the TriBody format retained the binding to NKG2D protein. Similarly, as shown in FIG. 12, the binding was also confirmed on cells over expressing both human and cyno NKG2D on the cell surface. Tribody IM-1245, IM-1246, IM-1249 and IM-1250 that represent mAb 001, mAb 009, mAb 010 and mAb 019, respectively, were further selected for functional activity in vitro.

### Example 7

### In Vitro Functional Evaluation of Tribody Antibody Constructs

Objective: To evaluate in vitro, dose dependent NK-cell activation by Tribody variants on primary PBMCs from healthy donor in the presence of cancer cells.

Methods: NK Cell Activation in the Presence of Target Cells. Suspend PBMCs with RPMI 1640 (Gibco, Cat-A10491-01) containing 10% FBS and 100 ng/ml hIL-2 (R&D SYSTEMS, cat-219-IL) for 2 days. After stimulation, harvest PBMCs and target cells NCI-H226 by centrifuging at 500 g for 5 min, adjust the concentration of cells to 4×10⁶/mL and 2×10⁵/mL respectively with assay buffer (RPMI 1640+5% FBS+10 ng/mL hIL-2), and then add 50 μL PBMC suspension and 50 μL NCI-H226 suspension respectively to a round-bottom 96 well plate with ET ratio of 20:1. Prepare diluted antibodies (2*) in assay buffer, add 100 μL/well, mix sufficiently. Add APC anti-CD107a (Biolegend, cat: 328620) and control (0.5 μL/test) into samples separately. Incubate the plate in a humidified 5% CO2 atmosphere at 37° C. for 4 hrs. After incubation, centrifuge the plate at 250 g for 4 min at 4° C., discard supernatant, and transfer all cells to a FACS plate using FACS buffer (PBS+2% FBS). After twice of wash with FCAS buffer, add FITC-anti-CD3, BV421-anti-CD56 and APC-CD107a within 100 μL FACS buffer, incubate the plate at 4° C. for 45 min in dark. After twice of wash with FCAS buffer, re-suspend cells with 100 μL cold PBS. Submit the cells for multi-color FACS analysis.

Results: 60-70% activation as measured by CD107a MFI on CD56⁺gated NK cells population was observed, following co-culturing PBMCs derived from healthy donor, and NCI-H226 target cells expressing 5T4 protein, in the presence of the various Tribodies (IM-1245, IM-1246, IM-1249, IM-1250) at 300 nM, 60 nM, 12 nM and 2.4 nM (left to right), while 30% basal activation in the absence of Tribody. Lower activation of 35-40% was observed in the negative control Tribody IM-1091 NKG2D mutant variant that is lack binding to NKG2D. IM-1060 served as a positive control Tribody (FIG. 13).

Conclusion: Four selected Tribodies that comprised of mouse anti human NKG2D mAbs clones sequences, from four bins were shown to be activating NK cells in vitro.

1. An isolated anti-NKG2D antibody comprising three complementarity determining regions (CDRs) on a heavy chain (HCDR1, HCDR2, and HCDR3) and three CDRs on a light chain (LCDR1, LCDR2, and LCDR3), wherein
(i) the HCDR1, HCDR2, and HCDR3 comprises the amino acid sequences of SEQ ID NOs:12-14, and the LCDR1, LCDR2, and LCDR3 comprises the amino acid sequences of SEQ ID NOs: 16-18; or
(ii) the HCDR1, HCDR2, and HCDR3 comprises the amino acid sequences of SEQ ID NOs:20-22, and the LCDR1, LCDR2, and LCDR3 comprises the amino acid sequences of SEQ ID NOs:24-26; or
(iii) the HCDR1, HCDR2, and HCDR3 comprises the amino acid sequences of SEQ ID NOs:28-30, and the LCDR1, LCDR2, and LCDR3 comprises the amino acid sequences of SEQ ID NOs:32-34; or
(iv) the HCDR1, HCDR2, and HCDR3 comprises the amino acid sequences of SEQ ID NOs:36-38, and the LCDR1, LCDR2, and LCDR3 comprises the amino acid sequences of SEQ ID NOs:40-42.

2. The anti-NKG2D antibody of claim 1, wherein the antibody comprises a heavy chain variable region and a light chain variable region, said heavy chain variable region and light chain variable region comprise the amino acid sequences of SEQ ID NOs: 11 and 15; SEQ ID NOs: 19 and 23; SEQ ID NOs:27 and 31; or SEQ ID NOs:35 and 39.

3. The anti-NKG2D antibody of claim 1, wherein the antibody is an IgG, a Fv, a scFv, a Fab, a F(ab′)₂, a minibody, a diabody, a triabody, a nanobody, a bispecific antibody, or a single domain antibody.

4. The anti-NKG2D antibody of claim 3, wherein said IgG is IgG1, IgG2, IgG3, or IgG4.

5. A composition comprising the anti-NKG2D antibody of claim 1 and a pharmaceutically acceptable carrier.

6. The composition of claim 5, wherein the anti-NKG2D antibody comprises three complementarity determining regions (CDRs) on a heavy chain (HCDR1, HCDR2, and HCDR3) and three CDRs on a light chain (LCDR1, LCDR2, and LCDR3), wherein
(i) the HCDR1, HCDR2, and HCDR3 comprises the amino acid sequences of SEQ ID NOs:12-14, and the LCDR1, LCDR2, and LCDR3 comprises the amino acid sequences of SEQ ID NOs: 16-18; or
(ii) the HCDR1, HCDR2, and HCDR3 comprises the amino acid sequences of SEQ ID NOs:20-22, and the LCDR1, LCDR2, and LCDR3 comprises the amino acid sequences of SEQ ID NOs:24-26; or
(iii) the HCDR1, HCDR2, and HCDR3 comprises the amino acid sequences of SEQ ID NOs:28-30, and the LCDR1, LCDR2, and LCDR3 comprises the amino acid sequences of SEQ ID NOs:32-34; or
(iv) the HCDR1, HCDR2, and HCDR3 comprises the amino acid sequences of SEQ ID NOs:36-38, and the LCDR1, LCDR2, and LCDR3 comprises the amino acid sequences of SEQ ID NOs:40-42.

7. The composition of claim 6, wherein the anti-NKG2D antibody comprises a heavy chain variable region and a light chain variable region, said heavy chain variable region and light chain variable region comprise the amino acid sequences of SEQ ID NOs: 11 and 15; SEQ ID NOs: 19 and 23; SEQ ID NOs:27 and 31; or SEQ ID NOs:35 and 39.

8. An isolated polynucleotide sequence encoding the anti-NKG2D antibody of claim 1.

9. A vector comprising the polynucleotide sequence of claim 8.

10. A host cell comprising the vector of claim 9.

11. A method of treating a disease in a subject, comprising the step of administering to the subject a composition comprising an effective amount of the anti-NKG2D antibody of claim 1.

12. The method of claim 11, wherein the disease is a cancer, an autoimmune disease, GvHD, a viral infection, a bacterial infection, or a disease associated with NKG2D.

13. The method of claim 11, wherein the anti-NKG2D antibody comprises three complementarity determining regions (CDRs) on a heavy chain (HCDR1, HCDR2, and HCDR3) and three CDRs on a light chain (LCDR1, LCDR2, and LCDR3), wherein
(i) the HCDR1, HCDR2, and HCDR3 comprises the amino acid sequences of SEQ ID NOs:12-14, and the LCDR1, LCDR2, and LCDR3 comprises the amino acid sequences of SEQ ID NOs: 16-18; or
(ii) the HCDR1, HCDR2, and HCDR3 comprises the amino acid sequences of SEQ ID NOs:20-22, and the LCDR1, LCDR2, and LCDR3 comprises the amino acid sequences of SEQ ID NOs:24-26; or
(iii) the HCDR1, HCDR2, and HCDR3 comprises the amino acid sequences of SEQ ID NOs:28-30, and the LCDR1, LCDR2, and LCDR3 comprises the amino acid sequences of SEQ ID NOs:32-34; or
(iv) the HCDR1, HCDR2, and HCDR3 comprises the amino acid sequences of SEQ ID NOs:36-38, and the LCDR1, LCDR2, and LCDR3 comprises the amino acid sequences of SEQ ID NOs:40-42.

14. The method of claim 13, wherein the anti-NKG2D antibody comprises a heavy chain variable region and a light chain variable region, said heavy chain variable region and light chain variable region comprise the amino acid sequences of SEQ ID NOs:11 and 15; SEQ ID NOs:19 and 23; SEQ ID NOs:27 and 31; or SEQ ID NOs:35 and 39.


SEQUENCE LISTING (42 sequences)

SEQ ID NO: 1 - protein, 121 aa, Artificial Sequence
  Description of Artificial Sequence Synthetic polypeptide
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 2 - protein, 9 aa, Artificial Sequence
  Description of Artificial Sequence Synthetic peptide
FTFSSYGMH

SEQ ID NO: 3 - protein, 14 aa, Artificial Sequence
  Description of Artificial Sequence Synthetic peptide
WVAFIRYDGSNKYY

SEQ ID NO: 4 - protein, 13 aa, Artificial Sequence
  Description of Artificial Sequence Synthetic peptide
KDRGLGDGTYFDY

SEQ ID NO: 5 - protein, 110 aa, Artificial Sequence
  Description of Artificial Sequence Synthetic polypeptide
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 6 - protein, 10 aa, Artificial Sequence
  Description of Artificial Sequence Synthetic peptide
SSNIGNNAVN

SEQ ID NO: 7 - protein, 11 aa, Artificial Sequence
  Description of Artificial Sequence Synthetic peptide
LLIYYDDLLPS

SEQ ID NO: 8 - protein, 10 aa, Artificial Sequence
  Description of Artificial Sequence Synthetic peptide
AAWDDSLNGP

SEQ ID NO: 9 - protein, 121 aa, Artificial Sequence
  Description of Artificial Sequence Synthetic polypeptide
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDAGEGDGTYFDYWGQGTTVTVSS

SEQ ID NO: 10 - protein, 110 aa, Artificial Sequence
  Description of Artificial Sequence Synthetic polypeptide
QSALTQPASVSGSPGQSITISCSGSSSNIGNNAVNWYQQLPGKAPKLLIYADDLLPSGVSDRFSGSKSGTSAFLAISGLQSEDEADYYCAAWDDSLNGPVFGGGTKLTVL

SEQ ID NO: 11 - protein, 123 aa, Artificial Sequence
  Description of Artificial Sequence Synthetic polypeptide
EVQLQQSGPELVKPGASVKISCKASAFNFTSYYLHWVKQGPGQGLEWIGWIYPGSGDTKYNEKFKGKATLTADTSSSAANMKLSSLTSEDSAVYYCARNRIFDSYYVDSMDYWGQGTSVTVSS

SEQ ID NO: 12 - protein, 10 aa, Artificial Sequence
  Description of Artificial Sequence Synthetic peptide
AFNFTSYYLH

SEQ ID NO: 13 - protein, 14 aa, Artificial Sequence
  Description of Artificial Sequence Synthetic peptide
WIGWIYPGSGDTKY

SEQ ID NO: 14 - protein, 15 aa, Artificial Sequence
  Description of Artificial Sequence Synthetic peptide
RNRIFDSYYVDSMDY

SEQ ID NO: 15 - protein, 111 aa, Artificial Sequence
  Description of Artificial Sequence Synthetic polypeptide
DIVLTQSPASLAVSLGQRAIISCKASQSVSFAGSNLMHWYQQKPGQQPKLLIYRASNLEVGVPTRFSGSGSGTDFTLNIHPVDEDDAATYYCQQSREYPFTFGSGTKLEIK

SEQ ID NO: 16 - protein, 12 aa, Artificial Sequence
  Description of Artificial Sequence Synthetic peptide
QSVSFAGSNLMH

SEQ ID NO: 17 - protein, 11 aa, Artificial Sequence
  Description of Artificial Sequence Synthetic peptide
LLIYRASNLEV

SEQ ID NO: 18 - protein, 8 aa, Artificial Sequence
  Description of Artificial Sequence Synthetic peptide
QQSREYPF

SEQ ID NO: 19 - protein, 122 aa, Artificial Sequence
  Description of Artificial Sequence Synthetic polypeptide
EVKLLQSGGGLVQPGGSLKLSCAASGIDLSRYWMSWVRRAPGKGLEWIGEINPDSSTINYAPSLKDKFIISRDNAKNTLSLQMRKVRSEDTALYFCIRRRYYGSSYWYFDVWGTGTTVTVSS

SEQ ID NO: 20 - protein, 9 aa, Artificial Sequence
  Description of Artificial Sequence Synthetic peptide
IDLSRYWMS

SEQ ID NO: 21 - protein, 15 aa, Artificial Sequence
  Description of Artificial Sequence Synthetic peptide
WIGEINPDSSTINYA

SEQ ID NO: 22 - protein, 15 aa, Artificial Sequence
  Description of Artificial Sequence Synthetic peptide
IRRRYYGSSYWYFDV

SEQ ID NO: 23 - protein, 112 aa, Artificial Sequence
  Description of Artificial Sequence Synthetic polypeptide
DVLMTQTPLSLPVSLGDQASISCRSSQSIVHTNGNTYLEWYLQKPGQSPKLLIYRVFNRFSGVPDRFSGSGSGTEFTLKISRVEAEDLGVYYCFQGSHVPFTFGAGTKLELK

SEQ ID NO: 24 - protein, 13 aa, Artificial Sequence
  Description of Artificial Sequence Synthetic peptide
QSIVHTNGNTYLE

SEQ ID NO: 25 - protein, 11 aa, Artificial Sequence
  Description of Artificial Sequence Synthetic peptide
LLIYRVFNRFS

SEQ ID NO: 26 - protein, 8 aa, Artificial Sequence
  Description of Artificial Sequence Synthetic peptide
FQGSHVPF

SEQ ID NO: 27 - protein, 122 aa, Artificial Sequence
  Description of Artificial Sequence Synthetic polypeptide
QVQLQQPGAELVKPGASVKLSCKASGYTFTTYWIQWIKQRPGQGLEWIGEIDPSDIFTNYNQKFKGKATLTVDTSSSTAYMQLSSLTSEDSAVYYCARGVYDGYYVGYFDVWGTGTTVTVSS

SEQ ID NO: 28 - protein, 9 aa, Artificial Sequence
  Description of Artificial Sequence Synthetic peptide
YTFTTYWIQ

SEQ ID NO: 29 - protein, 14 aa, Artificial Sequence
  Description of Artificial Sequence Synthetic peptide
WIGEIDPSDIFTNY

SEQ ID NO: 30 - protein, 14 aa, Artificial Sequence
  Description of Artificial Sequence Synthetic peptide
RGVYDGYYVGYFDV

SEQ ID NO: 31 - protein, 111 aa, Artificial Sequence
  Description of Artificial Sequence Synthetic polypeptide
DIVLTQSPASLAMSLGKRATISCRASESVSIMGTNLINWYQQKLGQPPKLLIYHASNLETGVPARFSGSGSRTDFTLTIDPVEEDDVAVYYCLQGRKIPRTFGGGTKLEIK

SEQ ID NO: 32 - protein, 12 aa, Artificial Sequence
  Description of Artificial Sequence Synthetic peptide
ESVSIMGTNLIN

SEQ ID NO: 33 - protein, 11 aa, Artificial Sequence
  Description of Artificial Sequence Synthetic peptide
LLIYHASNLET

SEQ ID NO: 34 - protein, 8 aa, Artificial Sequence
  Description of Artificial Sequence Synthetic peptide
LQGRKIPR

SEQ ID NO: 35 - protein, 120 aa, Artificial Sequence
  Description of Artificial Sequence Synthetic polypeptide
QVQLKESGPGLVAPSQSLSITCTVSGFSLTSYAISWFRQPPGKGLECLGVIWTGGGTNYNSVFKSRLSIIKDNSKSQVFLKMNSLQTDDTARYYCARKGDEGYWGYFDVWGTGTTVTVSS

SEQ ID NO: 36 - protein, 9 aa, Artificial Sequence
  Description of Artificial Sequence Synthetic peptide
FSLTSYAIS

SEQ ID NO: 37 - protein, 13 aa, Artificial Sequence
  Description of Artificial Sequence Synthetic peptide
CLGVIWTGGGTNY

SEQ ID NO: 38 - protein, 14 aa, Artificial Sequence
  Description of Artificial Sequence Synthetic peptide
ARKGDEGYWGYFDV

SEQ ID NO: 39 - protein, 107 aa, Artificial Sequence
  Description of Artificial Sequence Synthetic polypeptide
DILLTQSPAILSVSPGERVSFSCRASQSIGTSIHWYQQRTNVSPRLLIKYASESISGIPSRFSGSGSGTDFTLSINNVESEDIADYYCQHSNRWPLTFGAGTKLELK

SEQ ID NO: 40 - protein, 8 aa, Artificial Sequence
  Description of Artificial Sequence Synthetic peptide
QSIGTSIH

SEQ ID NO: 41 - protein, 11 aa, Artificial Sequence
  Description of Artificial Sequence Synthetic peptide
LLIKYASESIS

SEQ ID NO: 42 - protein, 8 aa, Artificial Sequence
  Description of Artificial Sequence Synthetic peptide
QHSNRWPL